(19) United States
(12) Patent Application Publication
(10) Pub. No.: US 2001/0044416 A1
(43) Pub. Date: Nov. 22, 2001

(54) Immunostimulatory nucleic acids for inducing a Th2 immune response

(76) Inventors: Michael J. McCluskie, Ottawa (CA); Heather L. Davis, Ottawa (CA)

Correspondence Address: Helen Lockhart, c/o Wolf, Greenfield & Sacks, P.C., Federal Reserve Plaza, 600 Atlantic Avenue, Boston, MA, 02210-2211, US

(21) Appl. No.: 09/768,012

(22) Filed: Jan. 22, 2001

Related U.S. Application Data
(60) Provisional application No. 60/177,461, filed on Jan. 20, 2000.

(51) Int. Cl. A61K 48/00; A61K 31/675; A61K 38/13; A61K 31/573

(52) U.S. Cl. 514/44; 514/9; 514/179; 514/110

(57) ABSTRACT

The invention relates to methods and products for inducing an immune response using immunostimulatory nucleic acids. In particular the immunostimulatory nucleic acids preferentially induce a Th2 immune response. The invention is useful for treating and preventing disorders associated with a Th1 immune response or for creating a Th2 environment for treating disorders that are sensitive to Th2 immune responses.

### PRIORITY OF THE INVENTION

This application claims priority under Title 35 §119(e), of U.S. application Ser. No. 60/177,461, filed Jan. 20, 2000, entitled IMMUNOSTIMULATORY NUCLEIC ACIDS FOR INDUCING A TH2 RESPONSE, the entire contents of which are incorporated herein by reference.

### FIELD OF THE INVENTION

The invention relates to methods and products for inducing an immune response and preferably a Th2 immune response. In particular the invention relates to the use of immunostimulatory nucleic acids that preferentially induce a Th2 immune response. The invention is useful inter alia for treating and preventing disorders associated with a Th1 immune response or disorders that are sensitive to a Th2 immune response.

### BACKGROUND OF THE INVENTION

The existence of functionally polarized T cell responses based on the profile of cytokines secreted by CD4+ T helper (Th) cells has been well established. In general, Th1 cells secrete interferon-gamma (IFN-γ), interleukin (IL)-2, and tumor necrosis factor-beta (TNFβ), and are important in macrophage activation, the generation of both humoral and cell-mediated immune responses and phagocyte-dependent protective responses. Th2 cells secrete IL-4, IL-5, IL-10, and IL-13 and are more important in the generation of humoral immunity, eosinophil activation, regulation of cell-mediated immune responses, control of macrophage function and the stimulation of particular Ig isotypes (Morel et al., 1998, Romagnani, 1999). Th1 cells generally develop following infections by intracellular pathogens, whereas Th2 cells predominate in response to intestinal nematodes. In addition to their roles in protective immunity, Th1 and Th2 cells are responsible for different types of immunopathological disorders. For example, Th1 cells predominate in organ specific autoimmune disorders, Crohn's disease, Helicobacter pylori-induced peptic ulcer, acute solid organ allograft rejection, and unexplained recurrent abortion, whereas Th2 cells predominate in Omenn's syndrome, systemic lupus erythematosus, transplantation tolerance, chronic graft versus host disease, idiopathic pulmonary fibrosis, and progressive systemic sclerosis, and are involved in triggering of allergic reactions (Romagnani 1999, Singh et al., 1999). Therefore, for both prophylactic and therapeutic purposes, depending on the particular disease, a preference for either Th1 or Th2 type responses exists.

In recent years, a number of studies have demonstrated the ability of unmethylated CpG dinucleotides (i.e., the cytosine is unmethylated) within the context of certain flanking sequences (CpG motifs) to stimulate both innate and specific immune responses. Such sequences are commonly found in bacterial DNA which is immunostimulatory. Similar immunostimulation is also possible with synthetic oligodeoxynucleotides (ODN) containing CpG motifs (CpG ODN). It has been demonstrated that CpG DNA can induce stimulation of B cells to proliferate and secrete immunoglobulin (Ig), IL-6 and IL-12, and to be protected from apoptosis (Krieg et al., 1995, Yi et al., 1996, Klinman et al., 1996). These effects contribute to the ability of CpG DNA to have adjuvant activity. In addition, CpG DNA enhances expression of class II MHC and B7 co-stimulatory molecules (Davis et al., 1998, Sparwasser et al., 1998), that leads to improved antigen presentation. Furthermore, CpG DNA also directly activates monocytes, macrophages and dendritic cells to secrete various cytokines and chemokines (Klinman et al., 1996, Sparwasser et al., 1998, Halpern et al., 1996) that can provide T-helper functions. These in vitro effects were believed to be specific to the unmethylated CpG motifs since they were not induced by methylated bacterial DNA or in general by ODN that do not contain unmethylated CpG motifs.

Immunization of animals against a variety of antigens delivered both parenterally and mucosally demonstrate that addition of CpG ODN induces more Th1-like responses as indicated by strong cytotoxic T lymphocytes (CTL), high levels of IgG2a antibodies, and predominantly Th1 cytokines (e.g., IL-12 and IFN-γ but not IL-4 or IL-5) (Klinman et al., 1996, Davis et al., 1998, Roman et al., 1997, Chu et al., 1997, Lipford et al., 1997, Weiner et al., 1997, McCluskie and Davis, 1998, 1999). In some circumstances, however, as outlined above, for immunization against certain diseases, a Th1 response is undesirable. For parenteral administration, aluminum precipitates (alum) may be added to antigens to augment Th2 immune responses, however alum is generally considered not suitable for delivery to mucosal surfaces. Cholera toxin (CT) is a potent Th2 mucosal adjuvant commonly used in animal models (Spangler 1992, Holmgren et al., 1992), however, it is considered to be too toxic for use in humans.

### SUMMARY OF THE INVENTION

The invention relates in some aspects to the discovery of compounds that induce a Th2 immune response. It has previously been demonstrated that oligonucleotides containing immunostimulatory CpG motifs (CpG ODN or CpG nucleic acids) are effective parenteral and mucosal adjuvants to protein antigens that induce Th1 immune responses. It has been discovered according to an aspect of the invention that oligonucleotides that do not contain immunostimulatory CpG motifs (non-CpG ODN), when administered by a mucosal route, augment immune responses and create a Th2 environment. The non-CpG ODN useful for producing these effects are referred to as Th2-immunostimulatory nucleic acids. These effects occur even with low doses of Th2 immunostimulatory nucleic acids. For instance, antibody levels are augmented almost as much as with CpG nucleic acids. While CpG nucleic acids push the immune responses in a Th1 direction, however, the Th2 immunostimulatory nucleic acids give a Th2-biased response. A “Th2 biased immune response” refers to the induction of at least one Th2-cytokine or an antibody typical of a Th2 response (Th2-antibody). This type of response was unexpected for several reasons. Th2 immunostimulatory nucleic acids do not induce this effect at typical adjuvant doses by parenteral routes. Nor do Th2 immunostimulatory nucleic acids have immune stimulatory effects in vitro that would predict such an in vivo response. It was also discovered that the Th2 immunostimulatory nucleic acids can produce an immune response such as an adjuvant effect with the administration of high doses by parenteral routes, or by direct delivery to affected tissues.

Thus one aspect of the invention is a method for inducing an antigen specific response by administering to a subject an antigen and a Th2-immunostimulatory nucleic acid in an amount effective to produce an antigen specific immune response when the Th2 immunostimulatory nucleic acid is administered mucosally or dermally. The effective amount is generally much lower than that required to induce an immune response when administered parenterally. Thus, in some embodiments, the effective dose ranges from 1 ng/kg to 1 mg/kg per administration. In other embodiments, the effective dose ranges from 0.01 μg/kg to 500 μg/kg per administration. In preferred embodiments, the range is from 0.1 μg/kg to 250 μg/kg per administration, in even more preferred embodiments, the range is from 1 μg/kg to 100 μg/kg per administration. In other embodiments, the mucosal or dermal effective amount ranges from 15 ng/kg to 150 μg/kg per administration, and in still others from 150 ng/kg to 15 μg/kg per administration. In some embodiments the Th2-immunostimulatory nucleic acid is delivered to the mucosa or locally to tissue such as the skin or eyeball. Although the Th2-immunostimulatory nucleic acid is administered mucosally or to the skin in some embodiments, it can produce a systemic immune response as well as a mucosal immune response. In certain embodiments, the dose of antigen administered along with the Th2 immunostimulatory nucleic acid is also lower than would be expected to be useful. In some embodiments doses of antigen which can effectively be used to induce an antigen specific immune response when administered with a Th2 immunostimulatory nucleic acid range from 0.1 μg to 10 μg total dose per administration, and in some instances from 1 μg to 100 μg total dose per administration. This range represents a 10-100 fold decrease over the amount of antigen which is required to induce an immune response when administered alone.

In another aspect of the invention, a method is provided for inducing an antigen specific response by administering to a subject an antigen and a Th2 immunostimulatory nucleic acid in an amount effective to produce an antigen specific immune response when the Th2 immunostimulatory nucleic acid is administered parenterally. The effective amount required for parenteral administration is greater than that which is effective for mucosal or dermal administration. Parenteral effective amounts range from 0.01 mg/kg to 1 mg/kg per administration, preferably when in a non-formulated form. If the Th2 immunostimulatory nucleic acids are formulated, and especially when they are formulated together with an antigen, the doses can be reduced in some instances to as low as 0.0001 mg/kg per administration. The immune response generated in this manner is a systemic immune response.

In the most preferred embodiments, the Th2 immunostimulatory nucleic acids are administered at doses not exceeding 1 mg/kg per administration, whether delivered mucosally or parenterally.

In certain embodiments of the foregoing aspects, the antigen is not conjugated to the Th2 immunostimulatory nucleic acid. In important embodiments, the antigen is not a self antigen, and it is not bacterial or a viral antigen.

According to another aspect of the invention a method for treating a non-autoimmune Th1-mediated disease in a subject is provided. The method includes administering to a subject a Th2-immunostimulatory nucleic acid in an amount effective to produce a Th2 immune response, when the Th2 immunostimulatory nucleic acid is administered mucosally or dermally.

Another aspect of the invention provides a method for treating autoimmune disease is a subject. The method comprises administering to a subject a Th2 immunostimulatory nucleic acid in an amount effective to produce a Th2 immune response, when the Th2 immunostimulatory nucleic acid is administered mucosally or dermally. In some embodiments the method also involves administering an antigen, such as, for instance a self-antigen, to the subject, for instance, to produce an immune hyporesponsive state. In important embodiments particularly those involving the treatment of Th1 mediated autoimmune disease, if the antigen is a self antigen, the antigen and Th2 immunostimulatory nucleic acid are not conjugated to each other.

Importantly, in some embodiments, the subject has not been exposed to a Th1 immunostimulatory nucleic acid. As an example, the subject in some embodiments, has not been exposed to a bacteria or a virus that carries a Th1 immunostimulatory nucleic acid. The subject may have been exposed to a parasite, such an extracellular parasite or an obligate intracellular parasite. Thus, in some embodiments, the subject does not have a bacterial or viral infection. In several aspects of the invention, the subject is not experiencing an immune response that is attributable to a Th1 immunostimulatory nucleic acid. Rather, in certain aspects, the subject is not experiencing an immune response attributable to a Th1 immunostimulatory nucleic acid because the subject has not been in contact with a Th1 immunostimulatory nucleic acid.

In other embodiments, the subject is administered a Th1 immunostimulatory nucleic acid following the administration of the Th2 immunostimulatory nucleic acid. In still other embodiments, the Th2 immunostimulatory nucleic acid is administered to a subject at risk of developing an extracellular infection. In important embodiments, the extracellular infections include those that colonize mucosal tissues and surfaces such as fungal and yeast infections that are sexually transmitted or that affect cancer patients receiving chemotherapy.

The T2 immunostimulatory nucleic acids may comprise phosphodiester or a phosphorothioate backbone. Importantly, immunization at the mucosal surface is not dependent upon backbone modification, and phosphodiester backbone nucleic acids are as effective as phosphorothioate backbone modifications for inducing an immune response. This is a surprising finding given that phosphorothioate backbone nucleic acids have been reported to be more efficient as parenterally administered vaccines.

The Th2 immune response induced according to the methods of the invention is not dependent upon conjugation of antigen and the Th2 immunostimulatory nucleic acid. Thus, the antigen and the nucleic acid may be conjugated to each other but this is not required. In some embodiments, it is preferred that the antigen and nucleic acid are not conjugated to each other. Thus, the antigen and the Th2-immunostimulatory nucleic acid may be administered simultaneously or separately. For instance, the antigen may be administered after the Th2-immunostimulatory nucleic acid or before the Th2-immunostimulatory nucleic acid. Additionally, the antigen and the Th2-immunostimulatory nucleic acid may be administered to the same or different sites in the subject and may be administered using the same or different delivery vehicles. For instance, in some embodiments the antigen is delivered to the mucosa or skin and in other embodiments the antigen is administered parenterally. In important embodiments, antigens may be administered in low doses, or alternatively, antigens with low antigenicity or immunogenicity may be used in the methods of the invention. Administration of low doses of antigen with a Th2 immunostimulatory nucleic acid, particularly when administered mucosally, surprisingly results in a Th2 immune response against the antigen, rather than a Th1 antigen specific immune response or antigen specific tolerance, both of which have been reported following low dose antigen administration. Antigens reported to have poor immunogenicity profiles include peptide antigens and tumor antigens. Additionally, the methods of the invention can be used to stimulate an immune response in subjects who are hyporesponsive to a particular antigen, such as for example, Hepatitis B surface antigen.

In some embodiments the method also includes administering a therapeutic agent to the subject. The therapeutic agent in some embodiments is a Th1 adjuvant, a Th2 adjuvant, a cytokine, and/or a drug for treating Th1 mediated disorders, such as, for instance an anti-psoriasis cream.

The Th2-immunostimulatory nucleic acid and/or antigen and/or therapeutic agent may be formulated and delivered to the subject in any manner known in the art. For instance in some embodiments it is formulated in a liquid solution, as a powder or in a bioadhesive polymer. In other embodiments the Th2-immunostimulatory nucleic acid is administered to the skin or a superficially located mucosal membrane using a needleless jet injection or particulate delivery system, scarification, and/or tines. In yet other embodiments the antigen and/or therapeutic agent is administered using a delivery system selected from the group consisting of a needleless delivery system, a scarification delivery system, and a tine delivery system.

In some aspects of the invention, the Th2-immunostimulatory nucleic acid is administered to the mucosa or skin. In some embodiments the Th2-immunostimulatory nucleic acid is administered orally, intranasally, by inhalation, rectally, vaginally, intradermally, intra-ocularly, intraepidermally, or transdermally.

In some embodiments of the invention the method is a method for treating or preventing a Th1 mediated disorder. The Th1 mediated disorder may be selected from the group consisting of an autoimmune disease, Helicobacter pylori-induced peptic ulcer, psoriasis, Th1 inflammatory disorder (provided it is not induced by the presence of bacterial or viral Th1 immunostimulatory nucleic acid), acute kidney allograft rejection, and unexplained recurrent abortion. The autoimmune disease in other embodiments is selected from the group consisting of rheumatoid arthritis, Crohn's disease, multiple sclerosis, systemic lupus erythematosus, autoimmune encephalomyelitis, myasthenia gravis, and insulin-dependent diabetes.

According to other embodiments the method is a method for inducing a local Th2 environment in the subject. The subject may have, for instance, a Th1 mediated skin disorder, and the local Th2 environment is induced in the skin.

The invention in other aspects relates to pharmaceutical compositions. One pharmaceutical composition of the invention includes a Th2-immunostimulatory nucleic acid and an antigen in a pharmaceutically acceptable carrier. The composition may optionally include a therapeutic agent.

Yet another pharmaceutical composition includes a Th2-immunostimulatory nucleic acid and an adjuvant, in a pharmaceutically acceptable carrier. This composition may also optionally include an antigen.

The Th2-immunostimulatory nucleic acid and/or the antigen and/or therapeutic agent are in some embodiments formulated together or separately in a delivery vehicle selected from the group consisting of bioadhesive polymers, cochleates, dendrimers, enteric-coated capsules, emulsomes, ISCOMs, liposomes, microspheres, nanospheres, polymer rings, proteosomes, and virosomes. In some embodiments the Th2-immunostimulatory nucleic acid and antigen and/or therapeutic agent are present in different delivery vehicles and in other embodiments they are in the same delivery vehicles.

When the composition or methods include a therapeutic agent, the therapeutic agent may be, in some embodiments, a Th1 adjuvant, a Th2 adjuvant, a cytokine, an anti-bacterial agent, an anti-fungal agent, an anti-parasitic agent, an anti-viral agent, or a drug for treating Th1 mediated disorders.

In some embodiments the Th1 adjuvant is a CpG nucleic acids, MF59, SAF, MPL, or QS21. In other embodiments the Th2 adjuvant is selected from the group consisting of adjuvants that creates a depot effect, adjuvants that stimulate the immune system, adjuvants that create a depot effect and stimulate the immune system and mucosal adjuvants. Adjuvants that creates a depot effect include but are not limited to alum; emulsion-based formulations including mineral oil, non-mineral oil, water-in-oil or oil-in-water-in oil emulsion, oil-in-water emulsions such as Seppic ISA series of Montanide adjuvants; and PROVAX. Adjuvants that stimulates the immune system include but are not limited to saponins purified from the bark of the Q. saponaria tree; poly[di(carboxylatophenoxy)phosphazene; derivatives of lipopolysaccharides, muramyl dipeptide and threonyl-muramyl dipeptide; OM-174; and Leishmania elongation factor. Adjuvants that create a depot effect and stimulate the immune system include but are not limited to ISCOMs; SB-AS2; SB-AS4; non-ionic block copolymers that form micelles such as CRL 1005; and Syntex Adjuvant Formulation.

Mucosal adjuvants include but are not limited to CpG nucleic acids, Bacterial toxins, Cholera toxin, CT derivatives, CT B subunit; CTD53; CTK97; CTK104; CTD53/K63; CTH54; CTN107; CTE114; CTE112K; CTS61F; CTS106; and CTK63, Zonula occludens toxin, zot, Escherichia coli heat-labile enterotoxin, Labile Toxin, LT derivatives, LT B subunit; LT7K; LT61F; LT112K; LT118E; LT146E; LT192G; LTK63; and LTR72, Pertussis toxin, PT-9K/129G; Toxin derivatives; Lipid A derivatives, MDP derivatives; Bacterial outer membrane proteins, outer surface protein A (OspA) lipoprotein of Borrelia burgdorferi, outer membrane protein of Neisseria meningitidis; Oil-in-water emulsions, Aluminum salts; and Saponins, ISCOMs, the Seppic ISA series of Montanide adjuvants, Montanide ISA 720; PROVAX; Syntext Adjuvant Formulation; poly[di(carboxylatophenoxy) phosphazene and Leishmania elongation factor.

Drugs for treating Th1 mediated disorders include but are not limited to anti-psoriasis creams, eye drops, nose drops, sulfasalazine, glucocorticoids, propylthiouracil, methimazole, ¹³¹I, insulin, IFN-β1a, IFN-β1b, copolymer 1 (i.e., MS), glucocorticoids (i.e., MS), ACTH, avonex, azathioprine, cyclophosphamide, UV-B, PUVA, methotrexate, calcipitriol, cyclophosphamide, OKT3, FK-506, cyclosporin A, azathioprine, and mycophenolate mofetil.

The invention in other aspects relates to an improved method of the type involving antigen dependent cellular cytotoxicity (ADCC) for stimulating an immune response in a subject. The improvement in the method involves administering to the subject a Th2 immunostimulatory nucleic acid in an effective amount for inducing ADCC. In some embodiments the subject has cancer or is at risk of developing cancer. In some embodiments a monoclonal antibody is also administered to the subject. Monoclonal antibodies include but are not limited to Rituxan, IDEC-C2B8, anti-CD20 Mab, Panorex, 3622W94, anti-EGP40 (17-1A) pancarcinoma antigen on adenocarcinomas Herceptin, anti-Her2, Anti-EGFr, BEC2, anti-idiotypic-GD₃ epitope, Ovarex, B43.13, anti-idiotypic CA125, 4B5, Anti-VEGF, RhuMAb, MDX-210, anti-HER-2, MDX-22, MDX-220, MDX-447, MDX-260, anti-GD-2, Quadramet, CYT-424, IDEC-Y2B8, Oncolym, Lym-1, SMART M195, ATRAGEN, LDP-03, anti-CAMPATH, ior t6, anti CD6, MDX-11, OV103, Zenapax, Anti-Tac, anti-IL-2 receptor, MELIMMUNE-2, MELIMMUNE-1, CEACIDE, Pretarget, NovoMAb-G2, TNT, anti-histone, Gliomab-H, GNI-250, EMD-72000, LymphoCide, CMA 676, Monopharm-C, ior egf/r3, ior c5, anti-FLK-2, SMART 1D10, SMART ABL 364, and ImmuRAIT-CEA.

In other embodiments radiation or chemotherapy is administered to the subject. Chemotherapies include but are not limited to Taxol, cisplatin, doxorubicin, and adriamycin.

The invention in other aspects is a pharmaceutical composition of a Th2 immunostimulatory nucleic acid in an effective amount for inducing ADCC and a monoclonal antibody. Monoclonal antibodies include but are not limited to Rituxan, IDEC-C2B8, anti-CD20 Mab, Panorex, 3622W94, anti-EGP40 (17-1A) pancarcinoma antigen on adenocarcinomas Herceptin, anti-Her2, Anti-EGFr, BEC2, anti-idiotypic-GD₃ epitope, Ovarex, B43.13, anti-idiotypic CA125, 4B5, Anti-VEGF, RhuMAb, MDX-210, anti-HER-2, MDX-22, MDX-220, MDX-447, MDX-260, anti-GD-2, Quadramet, CYT-424, IDEC-Y2B8, Oncolym, Lym-1, SMART M195, ATRAGEN, LDP-03, anti-CAMPATH, ior t6, anti CD6, MDX-11, OV103, Zenapax, Anti-Tac, anti-IL-2 receptor, MELIMMUNE-2, MELIMMUNE-1, CEACIDE, Pretarget, NovoMAb-G2, TNT, anti-histone, Gliomab-H, GNI-250, EMD-72000, LymphoCide, CMA 676, Monopharm-C, ior egf/r3, ior c5, anti-FLK-2, SMART 1D10, SMART ABL 364, and ImmuRAIT-CEA.

According to other aspects, the invention relates to a composition of a Th2 immunostimulatory nucleic acid having a phosphodiester backbone, formulated in a delivery vehicle selected from the group consisting of bioadhesive polymers, enteric-coated capsules, microspheres, nanospheres, and polymer rings. In important embodiments, the phosphodiester Th2 immunostimulatory nucleic acid is formulated for mucosal delivery.

Each of the limitations of the invention can encompass various embodiments of the invention. It is therefore anticipated that each of the limitations of the invention involving any one element or combination of elements can be included in each aspect of the invention.

### BRIEF DESCRIPTION OF THE SEQUENCE LISTING

SEQ ID NO: 1 is the nucleotide sequence of non-CpG ODN #1982.

SEQ ID NO: 2 is the nucleotide sequence of non-CpG ODN #2138.

SEQ ID NO: 3 is the nucleotide sequence of CpG ODN #1826.

SEQ ID NO: 4 is the nucleotide sequence of CpG ODN #2006.

### BRIEF DESCRIPTION OF THE DRAWINGS

FIG. 1 is a bar graph depicting the effect of different oligonucleotides on HBsAg-specific IgG titers. FIGS. 1a and 1b show data from an ELISA end-point dilution titer for HBsAg-specific antibodies (anti-HBs GMT) in plasma taken 1 week after final oral immunization (on days 0, 7 and 14) with HBsAg (100 μg) without adjuvant or in combination with CpG ODN (motif#1826, 100 μg), non-CpG ODN (motif#1982, 100 or 500 μg) or Cholera toxin (CT, 10 μg) for total IgG (FIG. 1a) or IgG1 (black bars) and IgG2a (hatched bars) isotypes (FIG. 1b).

FIG. 2 is a bar graph depicting the effect of different oligonucleotides on HBsAg-specific IgG titers. BALB/c mice were immunized by intramuscular (IM) injection with 1 μg HBsAg without adjuvant or with 10 μg of CpG ODN (motif #1826) or non-CpG ODN (motif #1982) and the ELISA end-point dilution titer for HBsAg-specific antibodies (anti-HBs), total IgG (FIG. 2a) or IgG1 (hatched bars) or IgG2a (grey bars) isotypes (FIG. 2b), in plasma taken 4 weeks after immunization is shown.

FIG. 3 is a bar graph depicting the effect of different oligonucleotides on TT-specific IgG titers. BALB/c mice were immunized by oral delivery on days 0, 7 and 14 with TT (100 μg) without adjuvant or in combination with CpG ODN (motif #1826, 100 μg), non-CpG ODN (motif#41982, 100 or 500 μg) or Cholera toxin (CT, 10 μg) and the ELISA end-point dilution titer for TT-specific antibodies (anti-TT GMT), total IgG (FIG. 3a) or IgG1 (hatched bars) or IgG2a (grey bars) isotypes (FIG. 3b), in plasma taken 1 week after final immunization are shown.

FIG. 4 is a bar graph depicting the effect of different oligonucleotides on FLUVIRAL®-specific IgG titers. BALB/c mice were immunized by oral delivery on days 0, 7 and 14 with FLUVIRAL® (50 μl, 1/10 human dose) without adjuvant or in combination with 10 μg of CpG ODN (motif #1826) or non-CpG ODN (motif #2138 or #1982) and the ELISA end-point dilution titer for FLUVIRAL®-specific antibodies (anti-FLUVIRAL® GMT), total IgG (FIG. 4a) or IgG1 (hatched bars) or IgG2a (grey bars) isotypes (FIG. 4b), in plasma taken 1 week after final immunization are shown.

FIG. 5 is a bar graph showing the effect of different oligonucleotides on FLUARIX®-specific IgG titers. BALB/c mice were immunized by intramuscular (IM) injection with FLUARIX® (50 μl, 1/10 human dose) without adjuvant or in combination with 50 μg of CpG ODN (motif #2006) or non-CpG ODN (motif #1982) and the ELISA end-point dilution titer for FLUARIX-specific antibodies (anti-FLUARIX®) in plasma taken 2 weeks after immunization is shown.

FIG. 6 is a graph depicting the effect of different oligonucleotides on antigen-specific IgG titers. BALB/c mice were immunized by oral delivery on days 0, 7 and 14 with a combination of HBsAg/TT/FLUVIRAL® (10 μg, 10 μg, 50 μl respectively) without adjuvant or in combination with 10 μg CpG ODN (motif #1826), or non-CpG ODN (motif #1982) and the ELISA end-point dilution titer for HBsAg-specific antibodies (FIG. 6a), TT-specific antibodies (FIG. 6b, HBsAg/TT/FLUVIRAL®, filled circles or single antigen TT, filled triangles), FLUVIRAL-specific antibodies (FIG. 6c, HBsAg/TT/FLUVIRAL®, filled circles or with a single antigen FLUVIRAL®, filled triangles) in plasma of individual mice taken 1 week after final immunization is shown. Other mice were immunized with TT or FLUVIRAL® with 10 μg CpG ODN (motif #1826). Horizontal bars represent the group geometric mean.

FIG. 7 is a graph depicting the effect of different oligonucleotides on antigen-specific IgG titers. BALB/c mice were immunized by oral delivery on days 0, 7 and 14 with a combination of HBsAg/TT/FLUVIRAL® (10 μg, 10 μg, 50 μl respectively) without adjuvant or in combination with 10 μg CpG ODN (motif #1826), or non-CpG ODN (motif #1982) and the ELISA end-point dilution titer for FLUVIRAL®-specific (FIG. 7a) or TT-specific (FIG. 7b) antibodies of IgG1 (grey bars) or IgG2a (black bars) isotypes in plasma taken 1 week after final immunization is shown.

FIG. 8 is a bar graph depicting the effect of different oligonucleotides on TT-specific IgG titers. BALB/c mice were immunized by intrarectal (FIG. 8a), intranasal (FIG. 8b), or oral (FIG. 8c) delivery on days 0, 7 and 14 with TT (10 μg) without adjuvant or in combination with CpG ODN (motif #1826, 100 μg), non-CpG ODN (motif #1982, 100 μg) or Cholera toxin (CT, 10 μg) and the ELISA end-point dilution titer for TT-specific antibodies in plasma of individual mice taken 1 week after final immunization is shown.

FIG. 9 is a bar graph depicting the effect of different oligonucleotides by intranasal delivery on TT-specific IgG titers. BALB/c mice were immunized by intranasal delivery on days 0, 7 and 14 with TT (10 μg) without adjuvant or in combination with CpG ODN (motif #1826, 10 or 100 μg) or non-CpG ODN (motif #1982, 100 μg) and the ELISA end-point dilution titer for TT-specific antibodies (anti-TT GMT), total IgG (FIG. 9a) or of IgG1 (grey bars) or IgG2a (hatched bars) isotypes (FIG. 9b) in plasma taken 1 week after final immunization is shown.

FIG. 10 is a bar graph depicting the effect of different oligonucleotides by oral delivery on TT-specific IgG titers. BALB/c mice were immunized by oral delivery on days 0, 7 and 14 with TT (10 μg) without adjuvant or in combination with CpG ODN (motif #1826, 10 or 100 μg) or non-CpG ODN (motif #1982, 10 or 100 μg) and the ELISA end-point dilution titer for TT-specific antibodies (anti-TT GMT) total IgG (FIG. 10a) or IgG1 (grey bars) or IgG2a (hatched bars) isotypes (FIG. 10b) in plasma taken 1 week after final immunization. Titers were defined as the highest plasma dilution resulting in an absorbance value two times that of non-immune plasma, with a cut-off value of 0.05.

FIG. 11 is a bar graph depicting the effect of different oligonucleotides on HBsAg-specific IgA titers. BALB/c mice were immunized by oral delivery on days 0, 7 and 14 with HBsAg (100 μg) without adjuvant or in combination with CpG ODN (motif #1826, 100 or 500 μg), or non-CpG ODN (motif #1982, 100 or 500 μg) and the ELISA end-point dilution titer for HBsAg-specific IgA antibodies (anti-HBs IgA) in saliva (FIG. 11a), vaginal washes (FIG. 11b) and lung washes (FIG. 11c) taken 1 week after final immunization and pooled for each group are shown.

FIG. 12 is a bar graph depicting the effect of different oligonucleotides on TT-specific IgA titers. BALB/c mice were immunized by oral delivery on days 0, 7 and 14 with TT (100 μg) without adjuvant or in combination with CpG ODN (motif #1826, 100 or 500 μg), non-CpG ODN (motif #1982, 100 or 500 μg) or Cholera toxin (CT, 10 μg) and the ELISA end-point dilution titer for TT-specific IgA antibodies (anti-TT IgA) in vaginal washes collected 1 week after final immunization and pooled for each group is shown.

FIG. 13 is a bar graph depicting the effect of different oligonucleotides on FLUVIRAL®-specific IgA titers. BALB/c mice were immunized by oral delivery on days 0, 7 and 14 with FLUVIRAL® (50 μl, 1/10 human dose) without adjuvant or in combination with 10 μg of CpG ODN (motif #1826) or non-CpG ODN (motif #2138) and the ELISA end-point dilution titer for FLUVIRAL®-specific IgA antibodies (anti-FLUVIRAL® IgA) for individual mice in lung washes (FIG. 13a), vaginal washes (FIG. 13b), and saliva (FIG. 13c) taken 1 week after final immunization is shown.

FIG. 14 is a graph depicting the effect of different oligonucleotides on antigen-specific IgA titers. BALB/c mice were immunized by oral delivery on days 0, 7 and 14 with a combination of HBsAg/TT/FLUVIRAL® (10 μg, 10 μg, 50 μl respectively) without adjuvant or in combination with 10 μg CpG ODN (motif #1826), or non-CpG ODN (motif #1982) and the ELISA end-point dilution titer for TT-specific IgA antibodies (FIG. 14a), HBsAg-specific IgA antibodies (FIG. 14b), and FLUVIRAL®-specific IgA antibodies in lung washes of individual mice taken 1 week after final immunization is shown.

### DETAILED DESCRIPTION OF THE INVENTION

The invention is based in part on the discovery that certain nucleic acid molecules, when administered to a subject, induce a Th2 biased immune response. It was previously known in the art that CpG containing nucleic acids produce a Th1 immune response, but it was believed that nucleic acids lacking a CpG do not produce an immune response. Surprisingly, it was discovered that control oligonucleotides, nucleic acids that do not include a CpG, actually do produce an immune response when administered in vivo but that the type of immune response differs from that produced by CpG containing nucleic acids.

As shown in the Examples below, mice were immunized by intramuscular (IM), oral, intranasal (IN) or intrarectal (IR) administration of one of three antigens: purified small envelope protein of the hepatitis B virus (S protein), which comprises hepatitis B surface antigen (HBsAg); tetanus toxoid (TT); or an influenza virus vaccine (FLUVIRAL®). Single or multiple antigen combinations were used either alone or with CpG nucleic acids or Th2 immunostimulatory nucleic acids as adjuvant. As shown previously, CpG nucleic acids augmented antigen-specific antibody responses with all routes, and this gave a much more Th1-biased response than was obtained with antigen alone. As also shown previously, non-CpG nucleic acids had no effect when given by a parenteral route (e.g., intramuscularly, IM) at normal parenteral doses. Antibody responses were essentially the same as those with antigen alone at these doses. However, surprisingly, when administered by any of the mucosal routes (including low dose administration) or at high doses through parenteral routes, the Th2 immunostimulatory nucleic acids did augment antibody responses, often as much as did the CpG nucleic acids, however the response was Th2-biased (IgG1>>IgG2a). This was particularly unexpected since in vitro data do not predict an immunostimulatory role for these Th2 immunostimulatory nucleic acids. This discovery has important implications for induction of immune responses where Th1 -type responses are undesirable or Th2-type responses are essential, and in the treatment of Th1 -associated disorders, as well as generally in the induction of antigen specific immune responses. Additionally, the invention provides methods for inducing mucosal immune responses, and systemic immune responses, particularly to antigens that are administered in low dose or which have a low immunogenicity.

The methods of the invention are intended for a wide range of subjects. The Th2 immunostimulatory nucleic acids are effective in subjects when used prophylactically or therapeutically. Additionally, the Th2 immunostimulatory nucleic acids are effective in subjects who have not been previously exposed to Th1 immunostimulatory nucleic acids. A subset of subjects having a bacterial or viral infection have been exposed to a Th1 immunostimulatory nucleic acid derived from the infecting bacteria or virus. Thus, the efficacy of the Th2 immunostimulatory nucleic acids in the methods of the invention are not dependent upon the presence of Th1 immunostimulatory nucleic acids. In some aspects, the invention intends that the Th2 immunostimulatory nucleic acids be used in the treatment of Th1 mediated disorders which are not associated with the presence of Th1 immunostimulatory nucleic acids, especially Th1 immunostimulatory nucleic acids derived from bacteria and viruses.

In other aspects of the invention, the Th2 immunostimulatory nucleic acids are not intended to reduce a pre-existing a Th1 immune response, but rather are intended to induce a Th2 immune response, irrespective of a down-regulation of a Th1 immune response. Some Th2 immunostimulatory nucleic acids are capable of inducing some level of Th1 immune response, thus in some instances, administration of a Th2 immunostimulatory nucleic acid will result in an up-regulation of both a Th2 and a Th1 immune response, albeit with a bias towards the Th2 immune response. It should be understood that in these latter instances administration of the Th2 immunostimulatory nucleic acids will result in increase and not decrease in the level of Th1 antibodies and cytokines over pre-administration levels.

Many of the methods provided by the invention involve mucosal or dermal administration of Th2 immunostimulatory nucleic acids at doses that have no effect when administered parenterally (e.g., intramuscularly, intravenously, intraperitoneally, subcutaneously, or by infusion). Other methods of the invention are capable of inducing Th2 immune responses when the Th2 immunostimulatory nucleic acids are administered parenterally at high doses. Thus, as used herein, the term “effective amount” is dependent upon the route of administration, with effective mucosal or dermal amounts being much lower than parenteral effective amounts.

Thus, in one aspect the invention is a method for inducing an antigen specific response by administering to a subject an antigen and a Th2-immunostimulatory nucleic acid in an amount effective to produce an antigen specific immune response.

The results of the experiments presented in the Examples show that Th2 immunostimulatory nucleic acids act as an effective adjuvant to induce immune responses against two different protein antigens (HBsAg, TT) as well as a killed split viral vaccine (FLUVIRAL®) when administered at typical adjuvant doses to the mucosal surfaces of the respiratory or gastrointestinal tracts. This effect was totally unexpected since non-CpG nucleic acids do not have such an effect when they are delivered by a parenteral route (e.g., IM injection) in amounts normally sufficient for CpG nucleic acids to induce an immune response (Davis et al., 1998), nor do they cause innate immune activation when added in vitro to cultures of peripheral blood mononuclear cells (Krieg et al., 1995). The Th2 immunostimulatory nucleic acids when administered to the mucosa were able to induce levels of antigen-specific IgG in the plasma as much as did CpG nucleic acids. Both nucleic acids were also as effective as CT, a strong conventional mucosal adjuvant that is highly effective in mice but too toxic for human use. Mucosal delivery of vaccines is particularly attractive since it offers: ease, low cost and safety of administration (e.g., orally, nasal drops or spray, inhalation, intrarectal, intravaginal or ocular administrations), thus removing the need for syringes and highly trained personnel; the generation of protective immunity at sites distant from the immunization site (Haneberg et al., 1994, Gallichan et al., 1995); no risk of needle stick injury or cross contamination through repeated use of the same needle, for example in poorer areas of the world; and, a broader age range of recipients (Walker et al., 1994).

Additionally, it was discovered that high doses of Th2 immunostimulatory nucleic acids administered in vivo are capable of provoking an immune response. This is surprising because it has been reported extensively in the literature that CpG nucleic acids induce an immune response through the presence of unmethylated CpG dinucleotides. Control nucleic acids without CpG motifs (i.e., lacking CpG dinucleotides or having CpG in which the C is methylated) have failed to produce immune responses at the doses tested. As a result, the investigators have concluded that the unmethylated CpG dinucleotide is essential. Additionally, in vitro studies using control nucleic acids have indicated that the unmethylated CpG was essential to the ability of the nucleic acid to induce an immune response. It has been discovered that high doses of non-CpG containing nucleic acids when administered in vivo have antigen-specific immune stimulating properties.

A “Th2 immunostimulatory nucleic acid” as used herein is a nucleic acid that does not contain an unmethylated CpG dinucleotide and that produces a Th2 immune response. An unmethylated CpG dinucleotide refers to an unmethylated cytosine within the dinucleotide. Thus, the Th2 immunostimulatory nucleic acid may be a nucleic acid that does not have any CpG dinucleotides. Additionally, the Th2 immunostimulatory nucleic acid is not T-rich or does not contain a poly T motif (i.e., a TTTT motif), a poly G motif (i.e., a GGGG motif), or a methylated CpG motif.

The Th2 immunostimulatory nucleic acids produce an immune response that is predominately Th2 in nature. A “Th2 immune response” as used herein refers to the induction of at least one Th2 cytokine or antibody typical of a Th2 response (Th2 antibody). In some embodiments more than one Th2-cytokine or Th2-antibody is induced, optionally in the absence of CTL, which are associated with Th1 responses. Thus the ability of a nucleic acid to produce a Th2 immune response can be assessed by determining if a Th2-cytokine or Th2-antibody is induced. This can be accomplished using routine screening. For instance, test nucleic acids can be administered alone or with antigen to mice or other animals, e.g., orally, and then the mouse or other animal can be screened for any changes in cytokine or antibody profiles. Some Th2 immunostimulatory nucleic acids are also capable of inducing a Th1 immune response, albeit at lower levels than the Th2 immune response induced.

Thus the induction of a Th2 response refers to the partial or complete induction of at least one Th2-cytokine or Th2-antibody or an increase in the levels of at least one Th2-cytokine or Th2-antibody. The term “cytokine” is used as a generic name for a diverse group of soluble proteins, factors, co-stimulatory molecules, and peptides which act as humoral regulators at nano- to picomolar concentrations and which, either under normal or pathological conditions, modulate the functional activities of individual cells and tissues. These cytokines also mediate interactions between cells directly and regulate processes taking place in the extracellular environment. Cytokines play a role in directing the T cell response. Helper (CD4+) T cells orchestrate the immune response of mammals through production of soluble factors that act on other immune system cells, including other T cells. Most mature CD4+ T helper cells express one of two cytokine profiles: Th1 or Th2. Examples of cytokines secreted by T cells or other immune cells that are associated with Th1 responses include IL-2, IL-12, IL-13, interferon-γ (γ-IFN), and TNFβ. The Th1 subset promotes delayed-type hypersensitivity, cell-mediated immunity, and immunoglobulin class switching to IgG₂ₐ. The Th2 subset induces humoral immunity by activating B cells, promoting antibody production, and inducing class switching to IgG₁ and IgE. Examples of Th2 cytokines include, but are not limited to IL-4, IL-5, IL-6, IL-9, IL-10, and IL-13. Th2-antibodies include but are not limited to IgG1 and IgE. Preferably the amount of Th2 antibodies generated by the Th2 immunostimulatory nucleic acids is the same or greater than the amount of Th1 antibodies generated. Some Th1 antibodies, such as IgG2a, may also be induced, but they will not be the predominant form of antibody.

The Th2 immunostimulatory nucleic acids can be double-stranded or single-stranded. Generally, double-stranded molecules are more stable in vivo, while single-stranded molecules have increased immune stimulating activity.

Th1 immunostimulatory nucleic acids, as used herein, refer to nucleic acids that induce primarily a Th1 immune response. Examples of Th1 immunostimulatory nucleic acids include nucleic acids containing at least one unmethylated CpG motif and/or nucleic acids that are T-rich. Th1 immunostimulatory nucleic acids are associated with some bacterial and viral strains. Infection by these microbes induces a Th1 immune response. A Th1 immune response is an immune response characterized by one or more Th1 cytokines or Th1 antibodies, as described herein.

The terms “nucleic acid” and “oligonucleotide” are used herein to mean multiple nucleotides (i.e. molecules comprising a sugar (e.g. ribose or deoxyribose) linked to a phosphate group and to an exchangeable organic base, which is either a substituted pyrimidine (e.g. cytosine (C), thymine (T) or uracil (U)) or a substituted purine (e.g. adenine (A) or guanine (G)). Substituted pyrimidines and purines include both naturally occurring and synthetic bases. As used herein, the terms refer to oligoribonucleotides as well as oligodeoxyribonucleotides. The terms shall also include polynucleosides (i.e. a polynucleotide minus the phosphate) and any other organic base containing polymer. Nucleic acid molecules can be obtained from existing nucleic acid sources (e.g. genomic or cDNA), but are preferably synthetic (e.g. produced by oligonucleotide synthesis).

The term Th2 immunostimulatory nucleic acid, however, does not encompass a plasmid expression vector. As used herein the terms a “Th2 immunostimulatory nucleic acid or oligonucleotide” and a “plasmid expression vector” are mutually exclusive. The terms “Th2 immunostimulatory nucleic acid or oligonucleotide” are used to refer to any Th2 immunostimulatory nucleic acid except for an expression vector. An expression vector as used herein is a nucleic acid molecule which includes at least a promoter and a gene encoding a peptide or peptide fragment and which is capable of expressing the peptide or peptide fragment in a cell. The plasmid expression vector includes a nucleic acid sequence encoding the peptide which is operatively linked to a gene expression sequence which directs the expression of the peptide within a eukaryotic cell. The gene expression sequence is any regulatory nucleotide sequence, such as a promoter sequence or promoter-enhancer combination, which facilitates the efficient transcription and translation of the peptide to which it is operatively linked. The gene expression sequence may, for example, be a mammalian or viral promoter, such as a constitutive or inducible promoter. Such constructs are well known to those of skill in the art. The Th2 immunostimulatory nucleic acid, however, does include plasmids and other vectors that are not expression vectors. That is, Th2 immunostimulatory nucleic acids include vectors that are not capable of expressing a peptide or peptide fragment. Th2 immunostimulatory nucleic acids, however, include plasmids and other vectors which cannot express a peptide or peptide fragment, i.e. plasmids which are partially or completely methylated of plasmids that are missing or have defective gene expression sequences or genes etc. In other embodiments, the Th2 immunostimulatory nucleic acids specifically exclude all vectors whether they are expression vectors or not.

In some embodiments the Th2 immunostimulatory nucleic acid is an oligonucleotide in the range of between 6 and 100 and more preferably between 6 and 50 nucleotides in size, and even more preferably 15-50 nucleotides in size. Alternatively, the Th2 immunostimulatory nucleic acid can be larger than 100 nucleotides in length.

The Th2 immunostimulatory nucleic acids may be a stabilized nucleic acid molecule. A “stabilized nucleic acid molecule” shall mean a nucleic acid molecule that is relatively resistant to in vivo degradation (e.g. via an exo- or endo-nuclease). Stabilization can be a function of length or secondary structure. Th2 immunostimulatory nucleic acids that are tens to hundreds of kbs long are relatively resistant to in vivo degradation. For shorter Th2 immunostimulatory nucleic acids, secondary structure can stabilize and increase their effect. For example, if the 3′ end of an oligonucleotide has self-complementarity to an upstream region, so that it can fold back and form a sort of stem loop structure, then the oligonucleotide becomes stabilized and therefore exhibits more activity.

Some stabilized nucleic acids of the instant invention have a modified backbone. Modification of the nucleic acid backbone with, for example, phosphorothioate linkages provides enhanced activity of the Th2 immunostimulatory nucleic acids, in some aspects of the invention, when administered in vivo, and protects the nucleic acid from degradation by intracellular exo- and endo-nucleases. In other aspects, the backbone of the Th2 immunostimulatory is less important, and a phosphodiester backbone Th2 immunostimulatory nucleic acid is as effective as a phosphorothioate backbone Th2 immunostimulatory nucleic acid. As an example, when administered mucosally or dermally according to some aspects of the invention, Th2 immunostimulatory nucleic acids comprising a phosphodiester backbone, are as effective as phosphorothioate backbone counter-parts, and have the additional characteristic of inducing less of a Th1 immune response in the process. Other modified oligonucleotides include phosphodiester modified oligonucleotides, combinations of phosphodiester and phosphorothioate oligonucleotides, methylphosphonate, methylphosphorothioate, phosphorodithioate, and combinations thereof. Each of these combinations and their particular effects on immune cells is discussed, with respect to CpG oligonucleotides, in more detail in PCT Published Patent Application No. WO98/18810 claiming priority to U.S. Ser. No. 08/738,652, filed on Oct. 30, 1996, the entire contents of which are hereby incorporated by reference. It is believed that these modified oligonucleotides may show more stimulatory activity due to enhanced nuclease resistance, increased cellular uptake, increased protein binding, and/or altered intracellular localization.

Other stabilized oligonucleotides include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Oligonucleotides which contain diol, such as tetraethyleneglycol or hexaethyleneglycol, at either or both termini have also been shown to be substantially resistant to nuclease degradation.

In some instances stabilized nucleic acids are preferred because they are less susceptible to degradation. Nucleic acids, however, with other backbones may also be effective, although in cases where the backbone is nuclease sensitive, some form of formulation or delivery system may be preferred to protect the nucleic acids. Thus when a less stable nucleic acid is delivered to a subject, it is preferred that the nucleic acid be associated with a vehicle that delivers it directly into the cell. Such vehicles are known in the art and include, for example, liposomes and gene guns.

The Th2 immunostimulatory nucleic acid is administered to the subject with an antigen or in some cases the subject is exposed to the antigen to induce an antigen specific immune response. The antigen exposure may be active, e.g., the deliberate administration to a subject in need of such treatment, or passive. Passive exposure may occur prior to or following administration of the Th2 immune response. As an example, some of the prophylactic methods provided by the invention involve administration of Th2 immunostimulatory nucleic acids to subjects not yet exposed to an antigen but perhaps at risk of such exposure. An antigen specific immune response is an immune response characterized by the production of antibody which has specificity for an antigen. The antigen specific immune response may be a systemic or a mucosal immune response. As shown in the experiments described herein the Th2 immunostimulatory nucleic acids when administered in conjunction with the antigen produce IgG1 and in some cases IgG2a that are specific for the particular antigen. These antibodies are characteristic of a systemic immune response. The IgG2a is associated with a Th1 immune response and the IgG1 is associated with a Th2 immune response. Th2 immunostimulatory nucleic acids produce higher levels of IgG1 than IgG2a antibodies.

In addition to inducing systemic immune responses the Th2 immunostimulatory nucleic acids are also effective as mucosal adjuvants with many forms of antigen, such as those for which CT has been shown to be an effective adjuvant. This includes, but is not limited to, recombinant proteins, synthetic peptides, and attenuated or killed whole pathogens. Thus, in addition to the induction of Th2-biased systemic immune responses, the Th2 immunostimulatory nucleic acids can also augment antigen-specific mucosal immunity (i.e., secretory IgA), which helps protect against infection by preventing the entry of pathogens at mucosal surfaces. Owing to the existence of a common mucosal immune system, immunization with Th2 immunostimulatory nucleic acids at one mucosal surface can protect against infection by pathogens that enter via other mucosal routes (e.g., an oral vaccine could protect against a sexually transmitted disease or a respiratory infection). Thus the Th2 immunostimulatory nucleic acids are capable of inducing mucosal immunity in remote sites as well as local sites. A “remote site” as used herein is a mucosal tissue that is located in a different region of the body than the mucosal tissue to which the Th2 immunostimulatory nucleic acids has been administered. For instance if the Th2 immunostimulatory nucleic acids is administered intranasally, a remote site would be the mucosal lining of the gut.

The Th2 immunostimulatory nucleic acids are administered to subjects. A “subject” as used herein is a human or vertebrate animal including but not limited to a dog, cat, horse, cow, pig, sheep, goat, chicken, primate, e.g., monkey, fish (aquaculture species), e.g. salmon, rat, and mouse.

The subject is exposed to the antigen. As used herein, the term “exposed to” refers to either the active step of contacting the subject with an antigen or the passive exposure of the subject to the antigen. The term “administered” when used in conjunction with an antigen refers to the active step of bringing the subject in contact with the antigen. Methods for the active exposure, or administration, of an antigen to a subject are well-known in the art. In general, an antigen is administered directly to the subject by any means such as intravenous, intramuscular, oral, transdermal, mucosal, intranasal, intratracheal, or subcutaneous administration. The antigen can be administered systemically, mucosally, or locally. Methods for administering the antigen and the Th2 immunostimulatory nucleic acids are described in more detail below. A subject is passively exposed to an antigen if an antigen becomes available for exposure to the immune cells in the body. A subject may be passively exposed to an antigen, for instance, by entry of a foreign pathogen into the body or by the development of a tumor cell expressing a foreign antigen on its surface. When a subject is passively exposed to an antigen, in some embodiments the Th2 immunostimulatory nucleic acid is an oligonucleotide of 8-100 nucleotides in length and/or has a phosphate modified backbone.

The methods in which a subject is passively exposed to an antigen can be particularly dependent on timing of administration of the Th2 immunostimulatory nucleic acids. For instance, in a subject at risk of developing an infectious disease the subject may be administered the Th2 immunostimulatory nucleic acid on a regular basis when that risk is greatest, i.e., after exposure to an infectious agent. Additionally the Th2 immunostimulatory nucleic acids may be administered to travelers before they travel to foreign lands where they are at risk of exposure to infectious agents, especially Th1 mediated infectious agents. Likewise the Th2 immunostimulatory nucleic acids may be administered to soldiers or civilians at risk of exposure to biowarfare to induce an immune response to the antigen when and if the subject is exposed to it. It is particularly preferred when the infectious agent induces an extracellular infection such as extracellular parasites or obligate intracellular parasites.

An “antigen” as used herein is a molecule capable of provoking an immune response. Antigens include but are not limited to cells, cell extracts, proteins, polypeptides, peptides, polysaccharides, polysaccharide conjugates, peptide mimics of polysaccharides, lipids, glycolipids, carbohydrates, viruses and viral extracts and muticellular organisms such as parasites and allergens. The term antigen broadly includes any type of molecule which is recognized by a host immune system as being foreign. Antigens include but are not limited to microbial antigens. The term “antigen” does not encompass self-antigens, which are defined below. Preferably, the antigens of the invention are not conjugated to the Th2 immunostimulatory nucleic acids, and thus the antigen and nucleic acid may be administered on different schedules and by different routes from each other. In some important embodiments, the antigen is administered in low doses (i.e., doses that would not induce an immune response if administered alone). In other embodiments, the antigen is one known to be minimally immunogenic.

A “microbial antigen” as used herein is an antigen of a microorganism and includes but is not limited to infectious virus, infectious bacteria, infectious parasites, infectious yeast, and infectious fungi. Such antigens include the intact microorganism as well as natural isolates and fragments or derivatives thereof and also synthetic compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art. Some microorganisms are associated with a Th1 -mediated disease and others are associated with a Th2-mediated disease. When the Th2 immunostimulatory nucleic acid is administered as an adjuvant in order to produce an antigen-specific immune response, it may be used against microorganisms that are associated with a Th1 or Th2 mediated disease, for the prevention and treatment of infection with those organisms. If the Th2 immunostimulatory nucleic acid is administered to a subject having an active bacterial or viral infection, the infection is preferably caused by a microbe not associated with a Th1 immunostimulatory nucleic acid.

An extracellular antigen as used herein is an antigen associated with an extracellular infection, preferably by a microbe that exists entirely extracellularly when in a host body and which also contains Th1 immunostimulatory nucleic acid. An example of an extracellular antigen is an antigen from a bacteria that contains Th1 immunostimulatory nucleic acids. Antigens that are not extracellular antigens, as described herein, are referred to as non-extracellular antigens. Non-extracellular antigens include, but are not limited to, tumor antigens or antigens derived from microbes that are not associated with a Th1 immunostimulatory nucleic acid. The methods of the invention generally intend to use in some aspects the Th2 immunostimulatory nucleic acids as adjuvants for extracellular antigens but preferably only when those extracellular antigens are not conjugated to the Th2 immunostimulatory antigens. Non-extracellular antigens are intended for use with the Th2 immunostimulatory nucleic acids of the invention, whether in a conjugated or non-conjugated form. In important embodiments, the non-extracellular antigens are not conjugated to the Th2 immunostimulatory nucleic acids.

Examples of virus that have been found in humans include but are not limited to: Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Both gram negative and gram positive bacteria serve as antigens in vertebrate animals. Such gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Examples of fungi include: Cryptococcus neoformans, Histoplasma capsulatum, Coccidioides immitis, Blastomyces dermatitidis, Chlamydia trachomatis, Candida albicans. Other infectious organisms (i.e., protists) include: Plasmodium such as Plasmodium falciparum, Plasmodium malariae, Plasmodium ovale, and Plasmodium vivax and Toxoplasma gondii.

Parasites include but are not limited to blood-borne and/or tissues parasites such as Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii.

Other medically relevant microorganisms have been described extensively in the literature, e.g., see C.G.A Thomas, Medical Microbiology, Bailliere Tindall, Great Britain 1983, the entire contents of which is hereby incorporated by reference.

Although many of the microbial antigens described above relate to human disorders, the invention is also useful for treating other non-human vertebrates. Non-human vertebrates are also capable of developing infections which can be prevented or treated with the Th2 immunostimulatory nucleic acids disclosed herein. For instance, in addition to the treatment of infectious human diseases, the methods of the invention are useful for treating infections of animals.

As used herein, the term “treat”, “treated”, or “treating” when used with respect to an infectious disease refers to a prophylactic treatment which increases the resistance of a subject (a subject at risk of infection) to infection with a pathogen or, in other words, decreases the likelihood that the subject will become infected with the pathogen as well as a treatment after the subject (a subject who has been infected) has become infected in order to fight the infection, e.g., reduce or eliminate the infection or prevent it from becoming worse.

Many vaccines for the treatment of non-human vertebrates are disclosed in Bennett, K. Compendium of Veterinary Products, 3rd ed. North American Compendiums, Inc., 1995. As discussed above, antigens include infectious microbes such as virus, bacteria, parasites, and fungi and fragments thereof, derived from natural sources or synthetically. Infectious virus of both human and non-human vertebrates, include retroviruses, RNA viruses and DNA viruses. This group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of other RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigens in vertebrate animals include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A,B,C,D,E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents (CHINA virus).

Each of the foregoing lists is illustrative, and is not intended to be limiting.

In addition to the use of the Th2 immunostimulatory nucleic acids to induce an antigen specific immune response in humans, the methods of the preferred embodiments are particularly well suited for treatment of non-human vertebrates. Non-human vertebrates which exist in close quarters and which are allowed to intermingle as in the case of zoo, farm and research animals are also embraced as subjects for the methods of the invention. Zoo animals such as the felid species including for example lions, tigers, leopards, cheetahs, and cougars; elephants, giraffes, bears, deer, wolves, yaks, non-human primates, seals, dolphins and whales; and research animals such as mice, rats, hamsters and gerbils are all potential subjects for the methods of the invention.

Birds such as hens, chickens, turkeys, ducks, geese, quail, and pheasant are prime targets for many types of infections. Hatching birds are exposed to pathogenic microorganisms shortly after birth. Although these birds are initially protected against pathogens by maternal derived antibodies, this protection is only temporary, and the bird's own immature immune system must begin to protect the bird against the pathogens. It is often desirable to prevent infection in young birds when they are most susceptible. It is also desirable to prevent against infection in older birds, especially when the birds are housed in closed quarters, leading to the rapid spread of disease. Thus, it is desirable to administer the Th2 immunostimulatory nucleic acid to birds to enhance an antigen-specific immune response when antigen is present.

An example of a common infection in chickens is chicken infectious anemia virus (CIAV). CIAV was first isolated in Japan in 1979 during an investigation of a Marek's disease vaccination break (Yuasa et al., 1979, Avian Dis. 23:366-385). Since that time, CIAV has been detected in commercial poultry in all major poultry producing countries (van Bulow et al., 1991, pp. 690-699) in Diseases of Poultry, 9th edition, Iowa State University Press).

CIAV infection results in a clinical disease, characterized by anemia, hemorrhage and immunosuppression, in young susceptible chickens. Atrophy of the thymus and of the bone marrow and consistent lesions of CIAV-infected chickens are also characteristic of CIAV infection. Lymphocyte depletion in the thymus, and occasionally in the bursa of Fabricius, results in immunosuppression and increased susceptibility to secondary viral, bacterial, or fungal infections which then complicate the course of the disease. The immunosuppression may cause aggravated disease after infection with one or more of Marek's disease virus (MDV), infectious bursal disease virus, reticuloendotheliosis virus, adenovirus, or reovirus. It has been reported that pathogenesis of MDV is enhanced by CIAV (DeBoer et al., 1989, p. 28 In Proceedings of the 38th Western Poultry Diseases Conference, Tempe, Ariz.). Further, it has been reported that CIAV aggravates the signs of infectious bursal disease (Rosenberger et al., 1989, Avian Dis. 33:707-713). Chickens develop an age resistance to experimentally induced disease due to CAA. This is essentially complete by the age of 2 weeks, but older birds are still susceptible to infection (Yuasa, N. et al., 1979 supra; Yuasa, N. et al., Arian Diseases 24, 202-209, 1980). However, if chickens are dually infected with CAA and an immunosuppressive agent (IBDV, MDV etc.) age resistance against the disease is delayed (Yuasa, N. et al., 1979 and 1980 supra; Bulow von V. et al., J. Veterinary Medicine 33, 93-116, 1986). Characteristics of CIAV that may potentiate disease transmission include high resistance to environmental inactivation and some common disinfectants. The economic impact of CIAV infection on the poultry industry is clear from the fact that 10% to 30% of infected birds in disease outbreaks die.

Vaccination of birds, like other vertebrate animals can be performed at any age. Normally, vaccinations are performed at up to 12 weeks of age for a live microorganism and between 14-18 weeks for an inactivated microorganism or other type of vaccine. For in ovo vaccination, vaccination can be performed in the last quarter of embryo development. The vaccine may be administered subcutaneously, by spray, orally, intraocularly, intratracheally, nasally, or by other mucosal delivery methods described herein. Thus, the Th2 immunostimulatory nucleic acid can be administered to birds and other non-human vertebrates using routine vaccination schedules and the antigen is administered after an appropriate time period as described herein.

Cattle and livestock are also susceptible to infection. Disease which affect these animals can produce severe economic losses, especially amongst cattle. The methods of the invention can be used to protect against infection in livestock, such as cows, horses, pigs, sheep, and goats.

Cows can be infected by bovine viruses. Bovine viral diarrhea virus (BVDV) is a small enveloped positive-stranded RNA virus and is classified, along with hog cholera virus (HOCV) and sheep border disease virus (BDV), in the pestivirus genus. Although, Pestiviruses were previously classified in the Togaviridae family, some studies have suggested their reclassification within the Flaviviridae family along with the flavivirus and hepatitis C virus (HCV) groups (Francki, et al., 1991).

BVDV, which is an important pathogen of cattle can be distinguished, based on cell culture analysis, into cytopathogenic (CP) and noncytopathogenic (NCP) biotypes. The NCP biotype is more widespread although both biotypes can be found in cattle. If a pregnant cow becomes infected with an NCP strain, the cow can give birth to a persistently infected and specifically immunotolerant calf that will spread virus during its lifetime. The persistently infected cattle can succumb to mucosal disease and both biotypes can then be isolated from the animal. Clinical manifestations can include abortion, teratogenesis, and respiratory problems, mucosal disease and mild diarrhea. In addition, severe thrombocytopenia, associated with herd epidemics, that may result in the death of the animal has been described and strains associated with this disease seem more virulent than the classical BVDVs.

Equine herpesviruses (EHV) comprise a group of antigenically distinct biological agents which cause a variety of infections in horses ranging from subclinical to fatal disease. These include Equine herpesvirus-1 (EHV-1), a ubiquitous pathogen in horses. EHV-1 is associated with epidemics of abortion, respiratory tract disease, and central nervous system disorders. Primary infection of upper respiratory tract of young horses results in a febrile illness which lasts for 8 to 10 days. Immunologically experienced mares may be reinfected via the respiratory tract without disease becoming apparent, so that abortion usually occurs without warning. The neurological syndrome is associated with respiratory disease or abortion and can affect animals of either sex at any age, leading to incoordination, weakness and posterior paralysis (Telford, E. A. R. et al., Virology 189, 304-316, 1992). Other EHV's include EHV-2, or equine cytomegalovirus, EHV-3, equine coital exanthema virus, and EHV-4, previously classified as EHV-1 subtype 2.

Sheep and goats can be infected by a variety of dangerous microorganisms including visna-maedi.

Primates such as monkeys, apes and macaques can be infected by simian immunodeficiency virus. Inactivated cell-virus and cell-free whole simian immunodeficiency vaccines have been reported to afford protection in macaques (Stott et al. (1990) Lancet 36:1538-1541; Desrosiers et al. PNAS USA (1989) 86:6353-6357; Murphey-Corb et al. (1989) Science 246:1293-1297; and Carlson et al. (1990) AIDS Res. Human Retroviruses 6:1239-1246). A recombinant HIV gp120 vaccine has been reported to afford protection in chimpanzees (Berman et al. (1990) Nature 345:622-625).

Cats, both domestic and wild, are susceptible to infection with a variety of microorganisms. For instance, feline infectious peritonitis is a disease which occurs in both domestic and wild cats, such as lions, leopards, cheetahs, and jaguars. When it is desirable to prevent infection with this and other types of pathogenic organisms in cats, the methods of the invention can be used to vaccinate cats to protect them against infection.

Domestic cats may become infected with several retroviruses, including but not limited to feline leukemia virus (FeLV), feline sarcoma virus (FeSV), endogenous type C oncornavirus (RD-114), and feline syncytia-forming virus (FeSFV). Of these, FeLV is the most significant pathogen, causing diverse symptoms, including lymphoreticular and myeloid neoplasms, anemias, immune mediated disorders, and an immunodeficiency syndrome which is similar to human acquired immune deficiency syndrome (AIDS). Recently, a particular replication-defective FeLV mutant, designated FeLV-AIDS, has been more particularly associated with immunosuppressive properties.

The discovery of feline T-lymphotropic lentivirus (also referred to as feline immunodeficiency) was first reported in Pedersen et al. (1987) Science 235:790-793. Characteristics of FIV have been reported in Yamamoto et al. (1988) Leukemia, December Supplement 2:204S-215S; Yamamoto et al. (1988) Am. J. Vet. Res. 49:1246-1258; and Ackley et al. (1990) J. Virol. 64:5652-5655. Cloning and sequence analysis of FIV have been reported in Olmsted et al. (1989) Proc. Natl. Acad. Sci. USA 86:2448-2452 and 86:4355-4360.

Feline infectious peritonitis (FIP) is a sporadic disease occurring unpredictably in domestic and wild Felidae. While FIP is primarily a disease of domestic cats, it has been diagnosed in lions, mountain lions, leopards, cheetahs, and the jaguar. Smaller wild cats that have been afflicted with FIP include the lynx and caracal, sand cat, and pallas cat. In domestic cats, the disease occurs predominantly in young animals, although cats of all ages are susceptible. A peak incidence occurs between 6 and 12 months of age. A decline in incidence is noted from 5 to 13 years of age, followed by an increased incidence in cats 14 to 15 years old.

Viral, bacterial, and parasitic diseases in fin-fish, shellfish or other aquatic life forms pose a serious problem for the aquaculture industry. Owing to the high density of animals in the hatchery tanks or enclosed marine farming areas, infectious diseases may eradicate a large proportion of the stock in, for example, a fin-fish, shellfish, or other aquatic life forms facility. Prevention of disease is a more desired remedy to these threats to fish than intervention once the disease is in progress. Vaccination of fish is the only preventative method which may offer long-term protection through immunity. Nucleic acid based vaccinations are described in U.S. Pat. No. 5,780,448 issued to Davis.

The fish immune system has many features similar to the mammalian immune system, such as the presence of B cells, T cells, lymphokines, complement, and immunoglobulins. Fish have lymphocyte subclasses with roles that appear similar in many respects to those of the B and T cells of mammals. Vaccines can be administered by immersion or orally.

Aquaculture species include but are not limited to fin-fish, shellfish, and other aquatic animals. Fin-fish include all vertebrate fish, which may be bony or cartilaginous fish, such as, for example, salmonids, carp, catfish, yellowtail, seabream, and seabass. Salmonids are a family of fin-fish which include trout (including rainbow trout), salmon, and Arctic char. Examples of shellfish include, but are not limited to, clams, lobster, shrimp, crab, and oysters. Other cultured aquatic animals include, but are not limited to eels, squid, and octopi.

Polypeptides of viral aquaculture pathogens include but are not limited to glycoprotein (G) or nucleoprotein (N) of viral hemorrhagic septicemia virus (VHSV); G or N proteins of infectious hematopoietic necrosis virus (IHNV); VP1, VP2, VP3 or N structural proteins of infectious pancreatic necrosis virus (IPNV); G protein of spring viremia of carp (SVC); and a membrane-associated protein, tegumin or capsid protein or glycoprotein of channel catfish virus (CCV).

Polypeptides of bacterial pathogens include but are not limited to an iron-regulated outer membrane protein, (IROMP), an outer membrane protein (OMP), and an A-protein of Aeromonis salmonicida which causes furunculosis, p57 protein of Renibacterium salmoninarum which causes bacterial kidney disease (BKD), major surface associated antigen (msa), a surface expressed cytotoxin (mpr), a surface expressed hemolysin (ish), and a flagellar antigen of Yersiniosis; an extracellular protein (ECP), an iron-regulated outer membrane protein (IROMP), and a structural protein of Pasteurellosis; an OMP and a flagellar protein of Vibrosis anguillarum and V. ordalii; a flagellar protein, an OMP protein, aroA, and purA of Edwardsiellosis ictaluri and E. tarda; and surface antigen of Ichthyophthirius; and a structural and regulatory protein of Cytophaga columnari; and a structural and regulatory protein of Rickettsia.

Polypeptides of a parasitic pathogen include but are not limited to the surface antigens of Ichthyophthirius. Typical parasites infecting horses are Gasterophilus spp.; Eimeria leuckarti, Giardia spp.; Tritrichomonas equi; Babesia spp. (RBC's), Theileria equi; Trypanosoma spp.; Klossiella equi; Sarcocystis spp.

Typical parasites infecting swine include Eimeria bebliecki, Eimeria scabra, Isospora suis, Giardia spp.; Balantidium coli, Entamoeba histolytica; Toxoplasma gondii and Sarcocystis spp., and Trichinella spiralis.

The major parasites of dairy and beef cattle include Eimeria spp., Cryptosporidium sp., Giardia sp., Toxoplasma gondii; Babesia bovis (RBC), Babesia bigemina (RBC), Trypanosoma spp. (plasma), Theileria spp. (RBC); Theileria parva (lymphocytes); Tritrichomonas foetus; and Sarcocystis spp.

The major parasites of raptors include Trichomonas gallinae; Coccidia (Eimeria spp.); Plasmodium relictum, Leucocytozoon danilewskyi (owls), Haemoproteus spp., Trypanosoma spp.; Histomonas; Cryptosporidium meleagridis, Cryptosporidium baileyi, Giardia, Eimeria; Toxoplasma.

Typical parasites infecting sheep and goats include Eimeria spp., Cryptosporidium sp., Giardia sp.; Toxoplasma gondii; Babesia spp. (RBC), Trypanosoma spp. (plasma), Theileria spp. (RBC); and Sarcocystis spp.

Typical parasitic infections in poultry include coccidiosis caused by Eimeria acervulina, E. necatrix, E. tenella, Isospora spp. and Eimeria truncata; histomoniasis, caused by Histomonas meleagridis and Histomonas gallinarum; trichomoniasis caused by Trichomonas gallinae; and hexamitiasis caused by Hexamita meleagridis. Poultry can also be infected Emeria maxima, Emeria meleagridis, Eimeria adenoeides, Eimeria meleagrimitis, Cryptosporidium, Eimeria brunetti, Emeria adenoeides, Leucocytozoon spp., Plasmodium spp., Hemoproteus meleagridis, Toxoplasma gondii and Sarcocystis.

Parasitic infections also pose serious problems in laboratory research settings involving animal colonies. Some examples of laboratory animals intended to be treated, or in which parasite infection is sought to be prevented, by the methods of the invention include mice, rats, rabbits, guinea pigs, nonhuman primates, as well as the aforementioned swine and sheep.

Typical parasites in mice include Leishmania spp., Plasmodium berghei, Plasmodium yoelii, Giardia muris, Hexamita muris; Toxoplasma gondii; Trypanosoma duttoni (plasma); Kiossiella muris; Sarcocystis spp. Typical parasites in rats include Giardia muris, Hexamita muris; Toxoplasma gondii; Trypanosoma lewisi (plasma); Trichinella spiralis; Sarcocystis spp. Typical parasites in rabbits include Eimeria sp.; Toxoplasma gondii; Nosema cuniculi; Eimeria stiedae, Sarcocystis spp. Typical parasites of the hamster include Trichomonas spp.; Toxoplasma gondii; Trichinella spiralis; Sarcocystis spp. Typical parasites in the guinea pig include Balantidium caviae; Toxoplasma gondii; Klossiella caviae; Sarcocystis spp.

The methods of the invention can also be applied to the treatment and/or prevention of parasitic infection in dogs, cats, birds, fish and ferrets. Typical parasites of birds include Trichomonas gallinae; Eimeria spp., Isospora spp., Giardia; Cryptosporidium; Sarcocystis spp., Toxoplasma gondii, Haemoproteus/Parahaemoproteus, Plasmodium spp., LeucocytozoonlAkiba, Atoxoplasma, Trypanosoma spp. Typical parasites infecting dogs include Trichinella spiralis; Isopora spp., Sarcocystis spp., Cryptosporidium spp., Hammondia spp., Giardia duodenalis (canis); Balantidium coli, Entamoeba histolytica; Hepatozoon canis; Toxoplasma gondii, Trypanosoma cruzi; Babesia canis, Leishmania amastigotes; Neospora caninum.

Typical parasites infecting feline species include Isospora spp., Toxoplasma gondii, Sarcocystis spp., Hammondia hammondi, Besnoitia spp., Giardia spp.; Entamoeba histolytica; Hepatozoon canis, Cytauxzoon sp., Cytauxzoon sp., Cytauxzoon sp. (red cells, RE cells).

Typical parasites infecting fish include Hexamita spp., Eimeria spp.; Cryptobia spp., Nosema spp., Myxosoma spp., Chilodonella spp., Trichodina spp.; Plistophora spp., Myxosoma Henneguya; Costia spp., Ichthyophithirius spp., and Oodinium spp.

Typical parasites of wild mammals include Giardia spp. (carnivores, herbivores), Isospora spp. (carnivores), Eimeria spp. (carnivores, herbivores); Theileria spp. (herbivores), Babesia spp. (carnivores, herbivores), Trypanosoma spp. (carnivores, herbivores); Schistosoma spp. (herbivores); Fasciola hepatica (herbivores), Fascioloides magna (herbivores), Fasciola gigantica (herbivores), Trichinella spiralis (carnivores, herbivores).

Parasitic infections in zoos can also pose serious problems. Typical parasites of the bovidae family (blesbok, antelope, banteng, eland, gaur, impala, klipspringer, kudu, gazelle) include Eimeria spp. Typical parasites in the pinnipedae family (seal, sea lion) include Eimeria phocae. Typical parasites in the camelidae family (camels, llamas) include Eimeria spp. Typical parasites of the giraffidae family (giraffes) include Eimeria spp. Typical parasites in the elephantidae family (African and Asian) include Fasciola spp. Typical parasites of lower primates (chimpanzees, orangutans, apes, baboons, macaques, monkeys) include Giardia sp.; Balantidium coli, Entamzoeba histolytica, Sarcocystis spp., Toxoplasma gondii; Plasmodim spp. (RBC), Babesia spp. (RBC), Trypanosoma spp. (plasma), Leishmania spp. (macrophages).

In addition to producing antigen-specific immune responses, the invention is also useful for inducing a Th2 immune response in a subject. When a subject is administered a Th2-immunostimulatory nucleic acid a Th2 immune response is produced. Thus, Th2 immunostimulatory nucleic acids can also be given on their own to establish a more Th2 environment or to treat Th1 -mediated disorders. Importantly, in some aspects, the Th1 mediated disorders are not those induced by the presence of Th1 immunostimulatory nucleic acids, especially those containing an unmethylated CpG dinucleotide, deriving from some bacterial and viral infections. Although Th1 mediated disorders display similar characteristics regardless of whether they are induced by the presence of microbial derived Th1 immunostimulatory nucleic acids or not, the invention intends to treat preferably only those of this latter category.

It was discovered according to the invention that Th2 immunostimulatory nucleic acids induced predominantly Th2-like responses (IgG1>>IgG2a), whereas CpG nucleic acids resulted in mixed Th1/Th2 or predominantly Th1-like responses. Th2 responses in some instances are also considered mixed immune response that are nonetheless biased towards a Th2 profile. Th2 responses are highly desirable for the prevention or treatment of a number of Th1-mediated diseases including: organ-specific autoimmune disorders, Crohn's disease, Helicobacter pylori-induced peptic ulcer, acute solid organ allograft rejection, and unexplained recurrent abortion. The only adjuvant currently licensed for use in humans in most countries of the world, including the USA, is aluminum hydroxide (alum) which, although having a Th2 immunostimulatory effect, is weak, is associated with undesirable local tissue reactions, and is generally considered unsuitable for mucosal delivery. CT, which also enhances Th2-like immune responses, can be given mucosally, however it is too toxic for use in humans. A mouse (˜20 g body weight) can tolerate the toxic effects of up to 10 μg of CT, however a dose as little as 1-5 μg will cause severe diarrhea in a human (˜70 kg body weight) (Jertborn et al., 1992). Animals receiving Th2 immunostimulatory nucleic acids showed no short-term signs of distress over those receiving antigen alone, and all recovered quickly with no apparent long-lasting effects even with doses of up to 500 μg. This is the first report of mucosal application of Th2 immunostimulatory nucleic acids to augment immune responses and the Th2-bias of the responses induced by Th2 immunostimulatory nucleic acids is of great importance in the development of effective Th2 biased prophylactic or therapeutic strategies.

Thus a subject, according to the invention, is a subject in need of a particular treatment. For instance, a subject may be a subject as risk of developing a disease such as cancer or an infectious disease or a subject that actually has cancer or an infectious disease. These subjects are administered the Th2 immunostimulatory nucleic acid of the invention, possibly in conjunction with an antigen to produce an antigen specific immune response to treat the cancer or infectious disease, thus preventing it from developing or from progressing, or alone to induce an antigen non-specific immune response.

Other subjects according to the invention are those that have or are at risk of developing a Th1 mediated disease. A “Th1 mediated disease” as used herein refers to a disease that is associated with the development of a Th1 immune response. A “Th1 immune response” as used herein refers to the induction of at least one Th1-cytokine or a Th1-antibody. In preferred embodiments more than one Th1-cytokine or Th1-antibody is induced. Thus a Th1-mediated disease is a disease associated with the induction of a Th1 response and refers to the partial or complete induction of at least one Th1-cytokine or Th1-antibody or an increase in the levels of at least one Th1-cytokine or Th1-antibody. These disorders are known in the art and include for instance, but are not limited to, autoimmune especially organ-specific autoimmune disease, psoriasis, Th1 inflammatory disorders, infection with extracellular parasites (e.g., response to helminths), solid organ allograft rejection (e.g., acute kidney allograft rejection), symptoms associated with hepatitis B (HBV) infection (e.g., HBV acute phase or recovery phase), chronic hepatitis C (HCV) infection, insulin-dependent diabetes mellitus (IDDM), multiple sclerosis (MS), “silent thyroiditis”, Crohn's disease, primary biliary cirrhosis, primary sclerosing cholangitis, sarcoidosis, atherosclerosis, acute graft versus host disease (GvHD), glomerulonephritis, anti-glomerular basement membrane disease, Wegener's granulomatosis, inflammatory myopathies, Sjögren's syndrome, Behget's syndrome, rheumatoid arthritis, Lyme arthritis, and unexplained recurrent abortion. Some Th1 mediated diseases and references where they are described are set forth below.

| | |
|---|---|
| Crohn's disease/IBD | Kakazu T et al., Type I T-helper cell predominance in granulomas of |
| | Crohnrs disease. Am J Gastroenterol 1999 Aug;94(8):2149-55; |
| | Monteleone G et al., Bioactive IL-18 expression is up-regulated in Crolin's |
| | disease. J Immunol 1999 Jul 1;163(1):143-7; |
| | Camoglio L et al., Altered expression of interferon-gamma and interleukin- |
| | 4 in inflammatory bowel disease. Inflamm Bowel Dis 1998 |
| | Nov;4(4):285-90; |
| | Plevy SE et al., A role for TNF-alpha and mucosal T helper-1 cytokines in |
| | the pathogenesis of Crohnts disease. J Immunol 1997 Dec |
| | 15;1 59(12):6276-82; |
| | Noguchi M et al., Enhanced interferon-gamma production and B7-2 |
| | expression in isolated intestinal mononuclear cells from patients with |
| | Crohnts disease. J Gastroenterol 1995 Nov;30 Suppl 8:52-5. |
| H. pylori | Hida N et al., Increased expression of IL-10 and IL-12 (p40) mRNA in |
| | Helicobacter pylori infected gastric mucosa: relation to bacterial cag |
| | status and peptic ulceration. J Clin Pathol 1999 Sep;52(9):658-64; |
| | Mattapallil JJ et al., A predominant Th1 type of immune response is |
| | induced early during acute Helicobacter pylori infection in rhesus |
| | macaques. Gastroenterology 2000 Feb;118(2):307-15. |
| Autoimmune | Okazaki K et al., Autoimmune-related pancreatitis is associated with |
| pancreatitis | autoantibodies and a Th1/Th2-type cellular immune response. |
| | Gastroenterology 2000 Mar;118(3):573-81. |
| Chronic hepatitis C | Bertoletti A et al., Different cytokine profiles of intraphepatic T cells in |
| | chronic hepatitis B and hepatitis C virus infections. Gastroenterology |
| | 1997 Jan;112(1):193-9; |
| | Quiroga JA et al., Induction of interleukin- 12 production in chronic |
| | hepatitis C virus infection correlates with the hepatocellular damage. J |
| | Infect Dis 1998 Jul;178(1):247-51. |
| Behcet's Syndrome | Sugi-Ikai N et al., Increased frequencies of interleukin-2- and interferon- |
| | gamma-producing T cells in patients with active Behcet's disease. Invest |
| | Ophthalmol Vis Sci 1998 May;39(6):996-1004. |
| PBC | Dienes HP et al., Bile duct epithelia as target cells in primary biliary |
| | cirrhosis and primary sclerosing cholangitis. Virchows Arch 1997 |
| | Aug;43 1(2): 119-24; |
| | Tjandra K et al., Progressive development of a Thi-type hepatic cytokine |
| | profile in rats with experimental cholangitis. Hepatology 2000 |
| | Feb;3 1(2):280-90; |
| | Harada K et al., In situ nucleic acid hybridization of cytokines in primary |
| | biliary cirrhosis: predominance of the Th1 subset. Hepatology 1997 |
| | Apr;25(4):79 1-6. |
| PSC | Dienes HP et al., Bile duct epithelia as target cells in primary biliary |
| | cirrhosis and primary sclerosing cholangitis. Virchows Arch 1997 |
| | Aug;43 1(2): 119-24; |
| | Tjandra K et al., Progressive development of a Th1-type hepatic cytokine |
| | profile in rats with experimental cholangitis. Hepatology 2000 |
| | Feb;31(2):280-90. |
| Sarcoidosis | Moller DR, Cells and cytokines involved in the pathogenesis of sarcoidosis. |
| | Sarcoidosis Vasc Difuse Lung Dis 1999 Mar;16(1):24-31; |
| | Moller DR et al., Enhanced expression of IL-12 associated with Th1 |
| | cytokine profiles in active pulmonary sarcoidosis. J Immunol 1996 Jun |
| | 15; 156(12):4952-60. |
| Atherosclerosis | Frostegard J et al., Cytokine expression in advanced human atherosclerotic |
| | plaques: dominance of pro-inflammatory (Th 1) and macrophage- |
| | stimulating cytokines. Atherosclerosis 1999 Jul; 145(1): 33-43. |
| Acute GvHD | Ochs LA et al., Cytokine expression in human cutaneous chronic graft- |
| | versus-host disease. Bone Marrow Transplant 1996 Jun;17(6):1085-92; |
| | Williamson B et al., Neutralizing IL-12 during induction of murine acute |
| | graft-versus-host disease polarizes the cytokine profile toward a Th2- |
| | type alloimmune response and confers long term protection from |
| | disease. J Immunol 1997 Aug 1;159(3): 1208-15. |
| Glomerulonephritis | Kitching AR et al., IFN-gamma mediates crescent formation and cell- |
| | mediated immune injury in murine glomerulonephritis. J Am Soc |
| | Nephrol 1999 Apr;10(4):752-9; |
| | Holdsworth SR et al., Th1 and Th2 T helper cell subsets affect patterns of |
| | injury and outcomes in glomerulonephritis. Kidney Int 1999 |
| | Apr;55(4):1198-216. |
| Wegener's | Gross WL et al., Pathogenesis of Wegener's granulomatosis. Ann Med |
| granulomatosis | Interne (Paris) 1998 Sep; 149(5):280-6. |
| Anti-GBM disease | Kalluri R et al., Susceptibility to anti-glomerular basement membrane |
| | disease and Goodpasture syndrome is linked to MHC class II genes and |
| | the emergence of T cell-mediated immunity in mice. J Clin Invest 1997 |
| | Nov 1;100(9):2263-75; |
| | Coelho SN et al., Immunologic determinants of susceptibility to |
| | experimental glomerulonepliritis: role of cellular immunity. Kidney Int |
| | 1997 Mar;51(3):646-52. |
| | Lepidi H et al., Local expression of cytokines in idiopathic inflammatory |
| | myopathies. Neuropathol Appl Neurobiol 1998 Feb;24(1): 73-9. |
| Siogren's syndrome | Kolkowski BC et al., Th1 predominance and perform expression in minor |
| | salivary glands from patients with primary Sjogren's syndrome. J |
| | Autoimmun 1999 Aug;13(1):155-62. |
| Lyme arthritis | Yin Z et al., T cell cytokine pattern in the joints of patients with Lyme |
| | arthritis and its regulation by cytokines and anticytokines. Arthritis |
| | Rheum 1997 Jan;40(1):69-79. |
| Rheumatoid arthritis | Kusaba M et al., Analysis of type 1 and type 2 T cells in synovial fluid and |
| | peripheral blood of patients with rheumatoid arthritis. J Rheumatol |
| | 1998 Aug;25(8):1466-71. |

As described above, when Th2 immunostimulatory nucleic acids are administered parenterally with antigen to produce an antigen-specific immune response, higher doses of the Th2 immunostimulatory nucleic acid are required than are required for mucosal administration. When the Th2 immunostimulatory nucleic acid is administered in combination with a therapeutic agent, higher doses are not required. Additionally, when the Th2 immunostimulatory nucleic acid is administered in order to induce a Th2 immune response or ADCC, higher doses are not required.

Autoimmune disease is a class of diseases in which an subject's own antibodies react with host tissue or in which immune effector T cells are autoreactive to endogenous self peptides and cause destruction of tissue. Thus an immune response is mounted against a subject's own antigens, referred to as self antigens. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

A “self-antigen” as used herein refers to an antigen of a normal host tissue. Normal host tissue does not include cancer cells. Thus an immune response mounted against a self-antigen, in the context of an autoimmune disease, is an undesirable immune response and contributes to destruction and damage of normal tissue, whereas an immune response mounted against a cancer antigen is a desirable immune response and contributes to the destruction of the tumor or cancer. Thus, in some aspects of the invention aimed at treating autoimmune disorders it is not recommended that the Th2 immunostimulatory nucleic acids be administered with self antigens, particularly those that are the targets of the autoimmune disorder.

A number of animal studies have demonstrated that mucosal administration of low doses of antigen can result in a state of immune hyporesponsiveness or “tolerance.” The active mechanism appears to be a cytokine-mediated immune deviation away from a Th1 towards a predominantly Th2 and Th3 (i.e., TGF-β dominated) response. The active suppression with low dose antigen delivery can also suppress an unrelated immune response (bystander suppression) which is of considerable interest in the therapy of autoimmune diseases, for example, rheumatoid arthritis and SLE. Bystander suppression involves the secretion of Th1 -counter-regulatory, suppressor cytokines in the local environment where proinflammatory and Th1 cytokines are released in either an antigen-specific or antigen-nonspecific manner. “Tolerance” as used herein is used to refer to this phenomenon. Indeed, oral tolerance has been effective in the treatment of a number of autoimmune diseases in animals including: experimental autoimmune encephalomyelitis (EAE) (Karpus et al., 1998, Rott et al., 1993, Chen et al., 1994), experimental autoimmune myasthenia gravis (Im et al., 1999, Ma et al., 1996), collagen-induced arthritis (CIA) (Nagler-Anderson et al., 1986), and insulin-dependent diabetes mellitus (Reddy et al., 2000, Ploix et al., 1998). In these models, the prevention and suppression of autoimmune disease is associated with a shift in antigen-specific humoral and cellular responses from a Th1 to Th2/Th3 response. Likewise, the Th2 immunostimulatory nucleic acids can also be used to promote Th2 responses in the treatment of multiple sclerosis and other Th1 -associated inflammatory disorders. This could be accomplished by the use of Th2 immunostimulatory nucleic acids on its own, or in association with a self-antigen (e.g., collagen for treatment of rheumatoid arthritis, or SLE, nuclear and nucleolar antigens for scleroderma).

The methods of the invention are also useful for preventing or treating disease associated with extracellular parasitic infections. Most parasites are host-specific or have a limited host range, i.e., they are able to infect a single or at most a few species. For example, P. yoelii is able to infect only rodents while P. falciparum and P. malariae are able to infect humans. The parasitic infection to be targeted by the methods and compounds of the invention will depend upon the host species receiving the prophylactic treatment and the conditions to which that host will become exposed.

Parasites can be classified based on whether they are intracellular or extracellular. An “intracellular parasite” as used herein is a parasite whose entire life cycle is intracellular. Examples of human intracellular parasites include Leishmania spp., Plasmodium spp., Trypanosoma cruzi, Toxoplasma gondii, Babesia spp., and Trichinella spiralis. An “extracellular parasite” as used herein is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites”. These parasites may exist most of their lives or only a small portion of their lives in an extracellular environment, but they all have at least one obligate intracellular stage in their life cycles. This latter category of parasites includes Trypanosoma rhodesiense and Trypanosoma gambiense, Isospora spp., Cryptosporidium spp, Eimeria spp., Neospora spp., Sarcocystis spp., and Schistosoma spp. The parasitic diseases which are classified as Th1 -mediated diseases of the invention include both extracellular parasites and obligate intracellular parasites which have at least one stage, and preferably more, of their life cycle that is extracellular. When the parasite is an extracellular parasite having at least one intracellular stage, the invention is useful for treating the parasite while it is in its extracellular stage, and, thus, when it is desirable to produce a Th2 environment.

In other aspects the method for inducing a Th2 immune response in a subject is useful for generating a Th2 environment. A “Th2 environment” as used herein is a local area of a subject that is characterized by the presence at least one type of Th2-cytokine or a Th2-antibody. Thus the generation of a Th2 environment is characterized by the induction of at least one type of Th2-cytokine or Th2-antibody. In some situations when it is desirable to generate a Th2 environment, the subject has a Th1 mediated disease but in other situations the subject may not have a Th1 mediated disease.

For example, ocular lesions are extremely common following HSV-1 reactivation and are associated with the infiltration of CD4+ and CD8+ T cells, macrophages, neutrophils and the production of Th 1 cytokines (Rouse, 1996). Thus, a treatment, according to the invention, is the topical administration of Th2 immunostimulatory nucleic acids capable of inducing Th2 cytokines. In a murine model of HSV infection, local treatment with or pre-exposure to Th2 cytokines (IL-10, IL-4, or TGF-β) but not Th1 cytokines (IL-2 or IFN-γ), reduced the severity of ocular lesions associated with HSV (Daheshia et al., 1997, 1998, Chun et al., 1998). Interestingly, intranasal delivery of TGF-β has also been shown to modulate the severity of ocular lesions caused by HSV infection (Kuklin et al., 1998).

The Th2 immunostimulatory nucleic acids may also be administered topically for the treatment of certain skin conditions. For example, the predominant mechanisms inducing skin lesions in psoriatic patients are thought to be interactions between infiltrating T cells and keratinocytes via the secretion of the Th1 cytokines IL-2 and IFN-γ the keratinocyte growth factor transforming growth factor alpha (TGF-α) and the cytokines IL-6 and IL-8. Several anti-psoriatic agents have been identified which act by selective stimulation of Th2 responses (De Jong et al., 1996, Ockenfels et al., 1998). Likewise, since it can selectively stimulate Th2 responses, Th2 immunostimulatory nucleic acids may also be a possible local treatment for Th1 mediated skin disorders.

The Th2 immunostimulatory nucleic acids may also be administered in conjunction with therapeutic agents, such as adjuvants. Therapeutic agents include but are not limited to systemic and mucosal adjuvants, Th1 or Th2 cytokines, anti-viral agents, anti-bacterial agents, anti-parasitic agents, anti-fungal, and drugs for treating Th1 mediated disorders. Therapeutic agents may be administered directly to the body or may be expressed from an expression system such as a plasmid vector or viral vector.

Immune responses can be induced and mediated with the co-administration of cytokines with the Th2 immunostimulatory nucleic acids. The term “cytokine” is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to IL-1, IL-2, IL-4, IL-5, IL-6, IL-7, IL-10, IL-12, IL-15, granulocyte-macrophage colony stimulating factor (GM-CSF), granulocyte colony stimulating factor (G-CSF), interferon-γ (γ-IFN), tumor necrosis factor (TNF), TGF-β, FLT-3 ligand, and CD40 ligand.

A systemic adjuvant is an adjuvant that can be delivered parenterally. Systemic adjuvants include adjuvants that creates a depot effect, adjuvants that stimulate the immune system and adjuvants that do both. An adjuvant that creates a depot effect as used herein is an adjuvant that causes the antigen to be slowly released in the body, thus prolonging the exposure of immune cells to the antigen. This class of adjuvants includes but is not limited to alum (e.g., aluminum hydroxide, aluminum phosphate); or emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720, AirLiquide, Paris, France); MF-59 (a squalene-in-water emulsion stabilized with Span 85 and Tween 80; Chiron Corporation, Emeryville, Calif.; and PROVAX (an oil-in-water emulsion containing a stabilizing detergent and a micelle-forming agent; IDEC, Pharmaceuticals Corporation, San Diego, Calif.).

Other adjuvants stimulate the immune system, for instance, cause an immune cell to produce and secrete cytokines or IgG. This class of adjuvants includes but is not limited to CpG nucleic acids, saponins purified from the bark of the Q. saponaria tree, such as QS21 (a glycolipid that elutes in the 21^{st} peak with HPLC fractionation; Aquila Biopharmaceuticals, Inc., Worcester, Me.); poly[di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

Other systemic adjuvants are adjuvants that create a depot effect and stimulate the immune system. These compounds are those compounds which have both of the above-identified functions of systemic adjuvants. This class of adjuvants includes but is not limited to ISCOMs (Immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxpropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

The mucosal adjuvants useful according to the invention are adjuvants that are capable of inducing a mucosal immune response in a subject when administered to a mucosal surface in conjunction with an antigen. Mucosal adjuvants include but are not limited to CpG nucleic acids (e.g. PCT published patent application WO 99/61056), Bacterial toxins: e.g., Cholera toxin (CT), CT derivatives including but not limited to CT B subunit (CTB) (Wu et al., 1998, Tochikubo et al., 1998); CTD53 (Val to Asp) (Fontana et al., 1995); CTK97 (Val to Lys) (Fontana et al., 1995); CTK104 (Tyr to Lys) (Fontana et al., 1995); CTD53/K63 (Val to Asp, Ser to Lys) (Fontana et al., 1995); CTH54 (Arg to His) (Fontana et al., 1995); CTN107 (His to Asn) (Fontana et al., 1995); CTE114 (Ser to Glu) (Fontana et al., 1995); CTE112K (Glu to Lys) (Yamamoto et al., 1997a); CTS61F (Ser to Phe) (Yamamoto et al., 1997a, 1997b); CTS106 (Pro to Lys) (Douce et al., 1997, Fontana et al., 1995); and CTK63 (Ser to Lys) (Douce et al., 1997, Fontana et al., 1995), Zonula occludens toxin, zot, Escherichia coli heat-labile enterotoxin, Labile Toxin (LT), LT derivatives including but not limited to LT B subunit (LTB) (Verweij et al., 1998); LT7K (Arg to Lys) (Komase et al., 1998, Douce et al., 1995); LT61F (Ser to Phe) (Komase et al., 1998); LT112K (Glu to Lys) (Komase et al., 1998); LT118E (Gly to Glu) (Komase et al., 1998); LT146E (Arg to Glu) (Komase et al., 1998); LT192G (Arg to Gly) (Komase et al., 1998); LTK63 (Ser to Lys) (Marchetti et al., 1998, Douce et al., 1997, 1998, Di Tommaso et al., 1996); and LTR72 (Ala to Arg) (Giuliani et al., 1998), Pertussis toxin, PT. (Lycke et al., 1992, Spangler BD, 1992, Freytag and Clemments, 1999, Roberts et al., 1995, Wilson et al., 1995) including PT-9K/129G (Roberts et al., 1995, Cropley et al., 1995); Toxin derivatives (see below) (Holmgren et al., 1993, Verweij et al., 1998, Rappuoli et al., 1995, Freytag and Clements, 1999); Lipid A derivatives (e.g., monophosphoryl lipid A, MPL) (Sasaki et al., 1998, Vancott et al., 1998; Muramyl Dipeptide (MDP) derivatives (Fukushima et al., 1996, Ogawa et al., 1989, Michalek et al., 1983, Morisaki et al., 1983); Bacterial outer membrane proteins (e.g., outer surface protein A (OspA) lipoprotein of Borrelia burgdorferi, outer membrane protine of Neisseria meningitidis)(Marinaro et al., 1999, Van de Verg et al., 1996); Oil-in-water emulsions (e.g., MF59) (Barchfield et al., 1999, Verschoor et al., 1999, O'Hagan, 1998); Aluminum salts (Isaka et al., 1998, 1999); and Saponins (e.g., QS21) Aquila Biopharmaceuticals, Inc., Worster, Me.) (Sasaki et al., 1998, MacNeal et al., 1998), ISCOMs, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micell-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene (PCPP polymer; Virus Research Institute, USA) and Leishmania elongation factor (Corixa Corporation, Seattle, Wash.).

Th2 adjuvants include most of the adjuvants listed above, except for CpG nucleic acids. Th1 adjuvants include CpG nucleic acids and MF59, SAF, MPL, and Q521 which under some circumstances, known in the art, induce Th1 -responses.

Drugs useful for treating Th1 mediated disorders include but are not limited to anti-psoriasis creams, eye or nose drops (e.g., containing cytokines) for herpetic stromal keratitis, Sulfasalazine (i.e., for treating Crohn's disease), glucocorticoids (i.e., Crohn's disease), propylthiouracil (i.e., Grave's disease), methimazole (i.e., Grave's disease), ¹³¹I (i.e., Grave's disease), and/or surgery (i.e., Grave's disease), insulin (i.e., IDDM), IFN-β1a (i.e., MS), IFN-β1b (i.e., MS), copolymer 1 (i.e., MS), glucocorticoids (i.e., MS), ACTH (i.e., MS), AVONEX (i.e., MS), glucocorticoids (i.e., pemphigus vulgaris), azathioprine (i.e., pemphigus vulgaris), cyclophosphamide (i.e., pemphigus vulgaris), glucocorticoids (i.e., psoriasis), UV-B (i.e., psoriasis), PUVA (i.e., psoriasis), methotrexate (i.e., psoriasis), calcipitriol (i.e., psoriasis), glucocorticoids (i.e., Sjöogren's syndrome), cyclophosphamide (i.e., Sjögren's syndrome), glucocorticoids (i.e., solid organ allograft rejection), OKT3 (i.e., solid organ allograft rejection), FK-506 (i.e., solid organ allograft rejection), cyclosporin A (i.e., solid organ allograft rejection), azathioprine (i.e., solid organ allograft rejection), mycophenolate mofetil (i.e., solid organ allograft rejection), and the following antipsoriatics: Acitretin; Anthralin; Azaribine; Calcipotriene; Cycloheximide; Enazadrem Phosphate; Etretinate; Liarozole Fumarate; Lonapalene; and Tepoxalin.

Antibacterial agents include but are not limited to Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmnenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafingin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin.

Anti-fungal agents include but are not limited to Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofingin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefungin; Sulconazole Nitrate; Terbinafine; Terconazole; Thiram; Ticlatone; Tioconazole; Tolciclate; Tolindate; Tolnaftate; Triacetin; Triafuigin; Undecylenic Acid; Viridoflilvin; Zinc Undecylenate; and Zinoconazole Hydrochloride.

Anti-parasitic agents include but are not limited to Acedapsone; Amodiaquine Hydrochloride; Amquinate; Arteflene; Chloroquine; Chloroquine Hydrochloride; Chloroquine Phosphate; Cycloguanil Pamoate; Enpiroline Phosphate; Halofantrine Hydrochloride; Hydroxychloroquine Sulfate; Mefloquine Hydrochloride; Menoctone; Mirincamycin Hydrochloride; Primaquine Phosphate; Pyrimethamine; Quinine Sulfate; and Tebuquine.

Anti-viral agents include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime.

When the Th2 immunostimulatory nucleic acid is administered in conjunction with antigens and/or therapeutics, the Th2 immunostimulatory nucleic acid can be administered before, after, and/or simultaneously with the antigens and/or therapeutics. For instance, the combination of Th2 immunostimulatory nucleic acid and/or therapeutic may be administered with a priming dose of antigen. Either or both of the Th2 immunostimulatory nucleic acid and/or therapeutic may then be administered with the boost dose. Alternatively, the combination of Th2 immunostimulatory nucleic acid and/or therapeutic may be administered with a boost dose of antigen. Either or both of the of Th2 immunostimulatory nucleic acid and/or therapeutic may then be administered with the prime dose. A “prime dose” is the first dose of antigen administered to the subject. In the case of a subject that has an infection the prime dose may be the initial exposure of the subject to the infectious microbe and thus the combination of Th2 immunostimulatory nucleic acid and/or therapeutic is administered to the subject with the boost dose. A “boost dose” is a second or third, etc, dose of antigen administered to a subject that has already been exposed to the antigen. In some cases the prime dose administered with the combination of Th2 immunostimulatory nucleic acid and/or therapeutic is so effective that a boost dose is not required to protect a subject at risk of infection from being infected. In cases where the combination of Th2 immunostimulatory nucleic acid and/or therapeutic is given without antigen with repeated administrations, the Th2 immunostimulatory nucleic acid and/or therapeutic may be given alone for one or more of the administrations.

Th2 immunostimulatory nucleic acids also increase antibody dependent cellular cytotoxicity (ADCC). ADCC can be performed using a Th2 immunostimulatory nucleic acid in combination with an antibody specific for a cellular target, such as a cancer cell. When the Th2 immunostimulatory nucleic acid is administered to a subject in conjunction with the antibody the subjects immune system is induced to kill the tumor cell. The antibodies useful in the ADCC procedure include antibodies which interact with a cell in the body. Many such antibodies specific for cellular targets have been described in the art and many are commercially available. These antibodies include but are not limited to those presented in the Table below.

Antibody-Based Immune Therapy Product Development (by companies)

| | | | | Clinical. Trial |
|---|---|---|---|---|
| Antibody | | | | Phase {tc \\|3 “Cl. |
| Classification | Indication | Drug Name/Antibody | Company(ies) | Trial Phase”} |
| 1 | non-Hodgkin's | Rituxan ™ (rituximab, | IDEC/Genentech, Inc./Hoffinann- | Mkt 12/97 (received |
| | lymphoma | Mabthera) (IDEC-C2B8, | La Roche (first monoclonal | mkt approval in EU |
| | | chimeric murine/human anti- | antibody licensed for the | June 98, CS) |
| | | CD2O MAb) | treatment of cancer in the U.S.) | |
| 1 | Adjuvant therapy | Panorex ® (17-1A) (murine | Centocor/Glaxo/Ajinomoto | III, expect results mid |
| | for colorectal | monoclonal antibody) | | 1998, est. NDA 2001, |
| | (Dukes-C) | | | on mkt in Germany |
| | | | | 1994 |
| 1 | Pancreatic, lung, | Panorex ® (17-1A) (chimeric | Centocor/Ajinomoto | III in U.S. and Europe |
| | breast, ovary | murine monoclonal antibody) | | |
| | non-small cell | 3622W94 MAb that binds to | Glaxo Wellcome plc | II (NCI Phase tin |
| | lung, prostate | EGP4O (17-lA) | | combo with IL-2 and |
| | (adjuvant) | pancarcinoma antigen on | | GM-CSF) |
| | | adenocarcinomas | | |
| 2 | Breast/ovarian | Herceptin, anti-Her2 bMAb | Genentech/Hoffmann-La Roche | FDA-approval |
| | | | | recommended |
| 2 | Renal cell | C225 (chimeric monoclonal | ImClone Systems | II/III (12/1997) |
| | | antibody to epidermal growth | | |
| | | factor receptor (EGFr)) | | |
| 2 | Breast | C225 (chimeric anti-EGFr | ImClone Systems | Ib/IIa (3/1996) |
| | | monoclonal antibody) + taxol | | |
| 2 | prostate | C225 (chimeric anti-EGFr | ImClone Systems (licensed from | Ib/IIa (1/1996) |
| | | monoclonal antibody ) + | RPR) | |
| | | doxorubicin | | |
| 2 | prostate | C225 (chimeric anti-EGFr | ImClone Systems | Ib/IIa (1/1996) |
| | | monoclonal antibody) + | | |
| | | adriamycin | | |
| 3 | Small cell lung | BEC2 (anti-idiotypic MAb, | ImClone Systems | III (5/1998) |
| | | mimics the GD3 epitope) | | |
| | | (with BCG?) | | |
| 3 | ? | Ovarex (B43.13, anti- | Altarex, Canada | II/III (1997) |
| | | idiotypic CA 125, mouse | | |
| | | MAb) | | |
| 3 | Melanoma | BEC2 (anti-idiotypic MAb, | ImClone Systems | Ib/IIa |
| | | mimics the GD3 epitope) | | |
| 3 | Melanoma, small- | 4B5 anti-idiotype Ab | Novopharm Biotech, Inc. | IND filed 9/1997 |
| | cell lung | | | |
| 4 | Lung, breast, | Anti-VEGF, RhuMAb | Genentech | II |
| | prostate, colorectal | (inhibits angiogenesis) | | |
| 5 | Breast, ovarian | MDX-210 (humanized anti- | Medarex/Novartis | II (6/1994) |
| | | HER-2 bispecific antibody) | | |
| 5 | Prostate, non- | MDX-210 (humanized anti- | Medarex/Novartis | II (5/1995) |
| | small cell lung, | HER-2 bispecific antibody) | | |
| | pancreatic, breast | | | |
| 5 | Renal and colon | MDX-210 (humanized anti- | Medarex/Novartis | II |
| | | HER-2 bispecific antibody) | | |
| 5 | Acute myleoid | MDX-22 (humanized | Medarex | II |
| | leukemia | bispecific antibody, MAb | | |
| | | conjugates) (complement | | |
| | | cascade activators) | | |
| 5 | Cancer | MDX-210 (humanized anti- | Medarex | I/II (7/1998) |
| | | HER-2 bispecific antibody) | | |
| 5 | Lung, colon, | MDX-220 (bispecific for | Medarex | I/II (1998) |
| | prostate, ovarian, | tumors that express TAG-72) | | |
| | endometrial, | | | |
| | pancreatic and | | | |
| | gastric | | | |
| 5 | Prostate | MDX-210 (humanized anti- | Medarex/Novartis | I/II (8/1996) |
| | | HER-2 bispecific antibody) | | |
| 5 | EGF receptor | MDX-447 (humanized anti- | Medarex/Merck KgaA | I/II (9/1995) |
| | cancers (bead & | EGF receptor bispecific | | |
| | neck, prostate, | antibody) | | |
| | lung, bladder, | | | |
| | cervical, ovarian) | | | |
| 5 | Comb. Therapy | MDX-210 (humanized anti- | Medarex/Novartis | I/II (6/1995) |
| | with G-CSF for | HER-2 bispecific antibody) | | |
| | various cancers, | | | |
| | esp. breast | | | |
| 5 | Melanoma, | MDX-260 bispecific, targets | Medarex, Inc. | Preclin. |
| | glioma, | GD-2 | | |
| | neuroblastoma | | | |
| | Bone metastases | Quadramet (CYT-424) | Cytogen Corp. | Submitted applic. For |
| | | radiotherapeutic agent | | approval in Canada |
| | | | | (3/1997), approved for |
| | | | | U.S. mkt? |
| | non-Hodgkin's | IDEC-Y2B8 (murine, anti- | IDEC | III |
| | lymhoma | CD20 MAb labeled with | | |
| | | Yttrium-90) | | |
| | non-Hodgkin's | Oncolym (Lym-1 monoclonal | Techniclone International/Alpha | II/III (1/1996) |
| | lymphoma | antibody linked to 131 iodine) | Therapeutics | |
| | Acute myleoid | SMART M195 Ab, | Protein Design Labs | II/III |
| | leukemia | humanized | | |
| | non-Hodgkin's | ¹³¹I LYM-1 (Oncolym ™) | Techniclone | II/III |
| | lymphoma | | Corporation/Cambridge Antibody | |
| | | | Technology | |
| | Acute | ATRAGEN ® | Aronex Pharmaceuticals, Inc. | II, to file NDA 1998 |
| | promyclocytic | | | |
| | leukemia | | | |
| | Head & neck, | C225 (chimeric anti-EGFr | ImClone Systems | II/III (1998) |
| | non-small cell | monoclonal antibody) + | | |
| | lung cancer | cisplatin or radiation | | |
| | non-Hodgkin's | Bexxar (anti-CD20 Mab | Coulter Pharma (Clinical results | II/III |
| | lymphoma | labeled with ¹³¹I) | have been positive, but the drug | |
| | | | has been associated with | |
| | | | significant bone marrow toxicity) | |
| | Kaposi's sarcoma | ATRAGEN ® | Aronex Pharmaceuticals, Inc. | II, completed |
| | B cell lymphoma | Rituxan ™ (MAb against | IDEC Pharmaceuticals | II (clinical trial in |
| | | CD20) pan-B Ab in combo. | Corp./Genentech | Germany underway) |
| | | with chemotherapy | | |
| | Chronic | LDP-03, huMAb to the | LeukoSite/Hex Oncology | II (1998) |
| | lymphocytic | leukocyte antigen | | |
| | leukemia (CLL) | CAMPATH | | |
| | Cancer | ior t6 (anti CD6, murine | Center of Molecular Immunology | IIb |
| | | MAb) CTCL | | |
| | Acute | MDX-11 (complement | Medarex | II (12/1993) |
| | myelogenous | activating receptor (CAR) | | |
| | leukemia (AML) | monoclonal antibody) | | |
| | Ex vivo bone | MDX-11 (complement | Medarex | II |
| | marrow purging in | activating receptor (CAR) | | |
| | acute | monoclonal antibody) | | |
| | myelogenous | | | |
| | leukemia (AML) | | | |
| | Ovarian | OV103 (Yttrium-90 labelled | Cytogen | II |
| | | antibody) | | |
| | Prostate | OV103 (Yttrium-90 labelled | Cytogen | II |
| | | antibody) | | |
| | non-Hodgkin's | ATRAGEN ® | Aronex Pharmaceuticals, Inc. | II |
| | lymphoma | | | |
| | Leukemia, | Zenapax (SMART Anti-Tac | Protein Design Labs | II |
| | lymphoma | (IL-2 receptor) Ab, | | |
| | | humanized) | | |
| | Acute | SMART M195 Ab, | Protein Design Labs | II |
| | promyclocytic | humanized | | |
| | leukemia | | | |
| | Melanoma | MELIMMUNE-2 (murine | IDEC | I/II (1993) |
| | | monoclonal antibody | | |
| | | therapeutic vaccine) | | |
| | Melanoma | MELIMMUNE-1 (murine | IDEC | I/II |
| | | monoclonal antibody | | |
| | | therapeutic vaccine) | | |
| | Colorectal and | CEACIDE ™ (1-131) | Immunomedics, Inc. | I/II |
| | other | | | |
| | non-Hodgkin's B | Pretarget ™ radioactive | NeoRx | I(6/1998) |
| | cell lymphoma | antibodies | | |
| | Cancer | NovoMAb-G2 (pancarcinoma | Novopharm Biotech, Inc. | I in Canada (12/97) |
| | | specific Ab) | | |
| | Brain | TNT (chimeric MAb to | Techniclone | I (11/97) |
| | | histone antigens) | Corporation/Cambridge Antibody | |
| | | | Technology | |
| | Brain | TNT (chimeric MAb to | Techniclone | I (11/1997) |
| | | histone antigens) | International/Cambridge | |
| | | | Antibody Technology | |
| | Brain, melanomas, | Gliomab-H (Monoclonals - | Novopharm | I (1/1996) |
| | neuroblastomas | Humanized Abs) | | |
| | Colorectal | GNI-250 MAb | Genetics Institute/AHP | I (>1991) |
| | Cancer | EMD-72000 (chimeric-EUF | Merck KgaA | I |
| | | antagonist) | | |
| | non-Hodgkin's B- | LymphoCide (humanized | Immunomedics | I |
| | cell lymphoma | LL2 antibody) | | |
| | Acute | CMA 676 (monoclonal | Immunex/AHP | I |
| | myelogenous | antibody conjugate) | | |
| | leukemia | | | |
| | Colon, lung, | Monopharm-C | Novopharm Biotech, Inc. | I |
| | pancreatic | | | |
| | Radioimmuno- | egf/r3 (anti EGF-R | Center of Molecular Immunology | IND filed |
| | therapy | humanized Ab) | | |
| | Colorectal | br c5 (murine MAb | Center of Molecular Immunology | IND filed |
| | | colorectal) for | | |
| | | radioimmunotherapy | | |
| | Breast cancer | BABS (biosynthetic antibody | Creative BioMolecules/Chiron | Lead/Preclin. |
| | | binding site) | | |
| | | proteins | | |
| | Tumor-associated | FLK-2 (monoclonal antibody | ImClone Systems/Chugai | Lead (1994) |
| | angiogenesis | to fetal liver kinase-2 (FLK- | | |
| | | 2)) | | |
| | Small-cell lung | Humanized MAb/small-drug | ImmunoGen, Inc. | Preclin. |
| | | conjugate | | |
| | Cancer | ANA Ab | Procyon Biopharma, Inc. | Preclin. |
| | B-cell lymphoma | SMART ID10 Ab | Protein Design Labs | Preclin. |
| | Breast, lung, colon | SMART ABL 364 Ab | Protein Design Labs/Novartis | Preclin. |
| | Colorectal | ImmuRAIT-CEA | Immunomedics, Inc. | Pilot clinicals |

In some embodiments of the invention, the Th2 immunostimulatory nucleic acids are administered to a subject having cancer, or a subject at risk of developing cancer in combination with a therapeutic agent, such as a chemotherapeutic agent. Chemotherapeutic agents include methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MM1270, BAY 12-9566, RAS famesyl transferase inhibitor, famesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZDO101, IS1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Placlitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan, cyclophosphamide, Aminoglutethimide, Asparaginase, Busulfan, Carboplatin, Chlorombucil, Cytarabine HCI, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate.

Th2 immunostimulatory nucleic acids may also be administered with cancer vaccines selected from the group consisting of EGF, Anti-idiotypic cancer vaccines, Gp75 antigen, GMK melanoma vaccine, MGV ganglioside conjugate vaccine, Her2/neu, Ovarex, M-Vax, O-Vax, L-Vax, STn-KHL theratope, BLP25 (MUC-1), liposomal idiotypic vaccine, Melacine, peptide antigen vaccines, toxin/antigen vaccines, MVA-based vaccine, PACIS, BCG vacine, TA-HPV, TA-CIN, DISC-virus and ImmuCyst/TheraCys. Biological response modifiers include interferon, and lymphokines such as IL-2. Hormone replacement therapy includes tamoxifen alone or in combination with progesterone.

One category of subjects intended for treatment according to the methods of the invention include those that have a cancer or are at risk of developing a cancer selected from the group consisting of basal cell carcinoma, bladder cancer, bone cancer, brain and CNS cancer, breast cancer, cervical cancer, colon and rectum cancer, connective tissue cancer, esophageal cancer, eye cancer, kidney cancer, larynx cancer, liver cancer, lung cancer, Hodgkin's lymphoma, Non-Hodgkin's lymphoma, melanoma, myeloma, leukemia, oral cavity cancer (e.g., lip, tongue, mouth, and pharynx), ovarian cancer, pancreatic cancer, prostate cancer, rhabdomyosarcoma, skin cancer, stomach cancer, testicular cancer, and uterine cancer. In preferred embodiments, the cancer to be treated may be selected from the group consisting of esophageal cancer, eye cancer, larynx cancer, oral cavity cancer (e.g., lip, tongue, mouth, and pharynx), skin cancer, cervical cancer, colon and rectum cancer, eye cancer, melanoma, stomach cancer, and uterine cancer.

The Th2 immunostimulatory nucleic acids and/or antigens and/or therapeutics may be delivered to the subject using conventional mucosal, local or parenteral routes as long as higher doses are administered when parenteral routes are used. Preferred mucosal routes of administration include but are not limited to oral, intranasal, intratracheal, inhalation, ocular, vaginal, and rectal.

For oral administration, the compounds (i.e., Th2-immunostimulatory nucleic acid, antigen, other therapeutic agent) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline and/or buffers for neutralizing internal acid conditions.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt. The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The compounds may also be administered locally. Compounds are administered locally when they are delivered directly to the site of action. For instance, local administration, includes but is not limited to delivery to the skin to induce antigen-specific immune responses or Th1 mediated skin disorders and direct injection or implantation into the site of a tumor. One preferred form of local administration is direct injection into the site of a tumor for ADCC.

The compounds of the invention can be administered to the skin, e.g., topically in the form of a skin cream, by injection into the skin, or any other method of administration where access to the skin cells and/or target APCs by the compounds is obtained. In some embodiments, topical administration is preferred, due to the accessibility of the skin and the ease of application. One method for accomplishing topical administration includes transdermal administration, such as iontophoresis. Iontophoretic transmission can be accomplished by using commercially-available patches which deliver a compound continuously through unbroken skin for periods of hours to days to weeks, depending on the particular patch. This method allows for the controlled delivery of the compounds through the skin in relatively high concentrations. One example of an iontophoretic patch is the LECTRO PATCH™ sold by General Medical Company of Los Angeles, Calif. The patch provides dosages of different concentrations which can be continuously or periodically administered across the skin using electronic stimulation of reservoirs containing the inhibitors or activators. Transdermal administration also includes needleless delivery methods such as those described in U.S. Pat. No. 5,630,796 and PCT Published Patent application WO99/27961. A needleless syringe is an instrument that delivers a compound transdermally without a conventional needle that pierces the skin. Transdermal delivery also includes intradermal (delivery into the dermis or epidermis), percutaneuos and transmucosal administration. Transmucosal administration is local, for instance, when the compounds are administered by direct injection into the mucosal tissue, i.e., the compounds may be injected into the inside of the cheek. Scarification is scratching of the surface of the skin to break through the epidermal layer before applying the drug.

Topical administration also includes epidermal administration which involves the mechanical or chemical irritation of the outermost layer of the epidermis sufficiently to provoke an immune response to the irritant. The irritant attracts APCs to the site of irritation where they can then take up the inhibitor or activator. One example of a mechanical irritant is a tyne-containing device. Such a device contains tynes which irritate the skin and deliver the drug at the same time. For instance, the MONO VACC® manufactured by Pasteur Merieux of Lyon, France. The device contains a syringe plunger at one end and a tyne disk at the other. The tyne disk supports several narrow diameter tynes which are capable of scratching the outermost layer of epidermal cells. Chemical irritants include, for instance, keratinolytic agents, such as salicylic acid and can be used alone or in conjunction with mechanical irritants.

The compounds may be in a liquid form. Alternatively, the active compounds may be in powder form for constitution with a suitable vehicle, e.g., sterile pyrogen-free water, before use or used directly as a powder. A powder as used herein refers to any type of solid dosage form including but not limited to particles, such as crystallized product, lyophilized product, spray coated material etc.

The compounds, when it is desirable to deliver them parenterally, may be formulated for administration by injection, e.g., by bolus injection or continuous infusion. Injections can be e.g., intravenous, intradermal, subcutaneous, intramuscular, or intraperitoneal. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

The Th2 immunostimulatory nucleic acids and/or antigens and/or therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a Th2 immunostimulatory nucleic acid and/or antigen and/or therapeutic optionally included in a pharmaceutically-acceptable carrier. The term “pharmaceutically-acceptable carrier” means one or more compatible solid or liquid filler, dilutants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The particular administration routes selected for use in the methods of the invention will depend, of course, upon the particular adjuvants or antigen selected, the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed herein.

The Th2 immunostimulatory nucleic acid may be directly administered to the subject or may be administered in conjunction with a nucleic acid delivery complex. A “nucleic acid delivery complex” shall mean a nucleic acid molecule associated with (e.g. ionically or covalently bound to; or encapsulated within) a targeting means (e.g. a molecule that results in higher affinity binding to target cell (e.g. dendritic cell surfaces and/or increased cellular uptake by target cells). Examples of nucleic acid delivery complexes include nucleic acids associated with: a sterol (e.g. cholesterol), a lipid (e.g. a cationic lipid, virosome or liposome), or a target cell specific binding agent (e.g. a ligand recognized by target cell specific receptor). Preferred complexes may be sufficiently stable in vivo to prevent significant uncoupling prior to internalization by the target cell. However, the complex can be cleavable under appropriate conditions within the cell so that the nucleic acid is released in a functional form. In some embodiments it is preferred that the nucleic acids that are delivered parenterally are associated with a nucleic acid delivery complex. By targeting the nucleic acids directly to the site of action, lower effective doses of the immunostimulatory nucleic acids can be used. This is especially important for parenteral delivery.

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories. For treatment of a patient, depending on activity of the compound, manner of administration, purpose of the immunization (i.e., prophylactic or therapeutic), nature and severity of the disorder, age and body weight of the patient, different doses may be necessary. The administration of a given dose can be carried out both by single administration in the form of an individual dose unit or else several smaller dose units. Multiple administration of doses at specific intervals of weeks or months apart is usual for boosting the antigen-specific responses.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Other delivery systems useful for administering the Th2 immunostimulatory nucleic acids include, but are not limited to, bioadhesive polymers (Sha et al., 1999), cochleates (Gould-Fogerite et al, 1994, 1996), dendrimers (Kukowska-Latallo et al., 1996, Qin et al, 1998), enteric-coated capsules (Czerkinsky et al., 1987, Levine et al., 1987), emulsomes (Vancott et al., 1998, Lowell et al., 1997), ISCOMs (Mowat et al., 1993, Morein et al., 1999, Hu et al, 1998, Carlsson et al., 1991), liposomes (Childers et al., 1999, Michalek et al, 1989, 1992), microspheres (Gupta et al, 1998, Maloy et al., 1994, Eldridge et al, 1989), nanospheres (Roy et al., 1999), polymer rings (Wyatt et al., 1998), proteosomes (Lowell et al., 1988, 1996) and virosomes (Gluck et al., 1992, Mengiardi et al., 1995, Cryz et al., 1998).

The term “effective amount” of a Th2 immunostimulatory nucleic acid refers to the amount necessary or sufficient to realize a desired biologic effect. For example, an effective amount of a Th2 immunostimulatory nucleic acid for inducing mucosal immunity is that amount necessary to cause the development of IgA in response to an antigen after exposure to the antigen. The effective amount of a Th2 immunostimulatory nucleic acid for inducing systemic immunity is that amount necessary to cause the development of IgG1 or Th2 cytokines in response to an antigen after exposure to the antigen. Additionally the effective amount of a Th2 immunostimulatory nucleic acid for generating or inducing a Th2 immune response or a Th2 environment is that amount necessary to cause the development of or increase in IgG1 or other Th2 cytokines.

Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular Th2 immunostimulatory nucleic acid being administered, the antigen, the other therapeutic, the size of the subject, or the severity of the disease or condition. One of ordinary skill in the art can empirically determine the effective amount of a particular Th2 immunostimulatory nucleic acid and/or antigen and/or therapeutic agent without necessitating undue experimentation.

One important parameter for identifying the effective amount of a Th2 immunostimulatory nucleic acid is the route of delivery. It has been discovered according to the invention that Th2 immunostimulatory nucleic acids administered mucosally or locally are effective in dose ranges which are generally similar to doses of CpG nucleic acids administered through the same routes. Nucleic acids delivered in combination with antigen by parenteral routes generally require higher effective doses to induce antigen specific immune responses. The Th2 immunostimulatory nucleic acids, however, administered parenterally for the purpose of inducing a Th2 immune response or for increasing ADCC or for inducing an antigen specific immune response when the Th2 immunostimulatory nucleic acids are administered in combination with other therapeutic agents or in specialized delivery vehicles are effective in dose ranges which are generally similar to doses of CpG nucleic acids administered through the same routes. In some embodiments higher doses are preferred for parenteral delivery.

Subject doses of the compounds described herein for mucosal or local delivery typically range from about 0.1 μg to 10 mg per administration, which depending on the application could be given daily, weekly, or monthly and any other amount of time therebetween. More typically mucosal or local doses range from about 10 μg to 5 mg per administration, and most typically from about 100 μg to 1 mg, with 2-4 administrations being spaced days or weeks apart. More typically, immune stimulant doses range from 1 μg to 10 mg per administration, and most typically 10 μg to 1 mg, with daily or weekly administrations.

Subject doses of the compounds described herein for parenteral delivery for the purpose of inducing an antigen-specific immune response, wherein the compounds are delivered with an antigen but not another therapeutic agent can typically be 5 to 10,000 times higher than the effective mucosal dose for vaccine adjuvant or immune stimulant applications, and more typically 10 to 1,000 times higher, and most typically 20 to 100 times higher. In important embodiments, the parenteral dose does not exceed 1 mg/kg per administration. The Th2 immunostimulatory nucleic acids may be administered at even greater doses, for example, at doses approximating 700 mg (i.e., 10 mg/kg) per administration, however, it is recommended that such doses are not administered in a single bolus and are rather administered in a number of administrations or by a number of delivery routes.

Doses of the compounds described herein for parenteral delivery for the purpose of inducing a Th2 immune response or for increasing ADCC or for inducing an antigen specific immune response when the Th2 immunostimulatory nucleic acids are administered in combination with other therapeutic agents or in specialized delivery vehicles typically range from about 0.1 μg to 10 mg per administration, which depending on the application could be given daily, weekly, or monthly and any other amount of time therebetween. More typically parenteral doses for these purposes range from about 10 μg to 5 mg per administration, and most typically from about 100 μg to 1 mg, with 2-4 administrations being spaced days or weeks apart. In some embodiments, however, parenteral doses for these purposes may be used in a range of 5 to 10,000 times higher than the typical doses described above.

For any compound described herein the therapeutically effective amount can be initially determined from animal models. A therapeutically effective dose can also be determined from human data for CpG oligonucleotides which have been tested in humans (human clinical trials have been initiated) and for compounds which are known to exhibit similar pharmacological activities, such as other mucosal adjuvants, e.g., LT and other antigens for vaccination purposes, for the mucosal or local administration. Higher doses are required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

In yet another aspect, the invention provides methods for screening nucleic acids for Th2 immunostimulatory activity. Preferably, candidate nucleic acids are tested using the methods described in the Examples. Briefly these methods entail administering to a subject, preferably a murine subject, a nucleic acid optionally with an antigen. Immunoglobulin isotype levels are measured in the subject prior to and following administration of the nucleic acid, as described. In preferred embodiments, the subject does not have above normal levels of Th1 type antibodies or cytokines prior to exposure to the candidate nucleic acid. Nucleic acids that induce the production or increase the level of Th2 type antibodies or cytokines, regardless of their effect on Th1 type antibodies or cytokines level or production can be used as Th2 immunostimulatory nucleic acids. In preferred embodiments, the subject has not been exposed to an infectious agent, especially a bacteria or a virus that carries a Th1 immunostimulatory nucleic acid, and/or does not have an infection by one of these types of microbes.

The invention will be more fully understood by reference to the following examples. These examples, however, are merely intended to illustrate the embodiments of the invention and are not to be construed to limit the scope of the invention. The following examples and the related figures refer to the Th2-immunostimulatory nucleic acid as a non-CpG ODN. For purposes of this patent application the terms “Th2-immunostimulatory nucleic acid” and “non-CpG ODN” are used interchangeably and have the meaning set forth herein for the term “Th2-immunostimulatory nucleic acid.”

### EXAMPLES

MATERIALS AND METHODS:

Immunization of mice: All experiments were carried out using female BALB/c mice aged 6-8 weeks with 5-10 mice per experimental or control group. For all immunizations, mice were lightly anaesthetized with Halothane® (Halocarbon Laboratories, River Edge, N.J.).

Antigens: Plasma-derived HBV S protein (HBsAg, ad subtype, Genzyme Diagnostics, San Carlos, Calif.), recombinant HBsAg (ay subtype, Medix Biotech, Foster City, Calif.), formalin-inactivated tetanus toxoid (TT, Pasteur Merieux Connaught, Swiftwater, Pa.), or trivalent influenza virus vaccine (A/Sydney/5/97, A/Beijing/262/95, B/Harbin/7/94, FLUVIRAL®, Biochem Vaccines Inc., Laval, QC, or FLUARIX®, SmithKline Beecham Pharmaceuticals).

Adjuvants: Non-CpG ODN motifs #1982 (5′-TCCAGGACTTCTCTCAGGTT-3′) (SEQ ID NO: 1), #2138 (5′-TCCATGAGCTTCCTGAGCTT-3′) (SEQ ID NO: 2), as well as CpG ODN motifs #1826 (TCCATGACGTTCCTGACGTT) (SEQ ID NO: 3) and #2006 (5′-TCGTCGTTTTGTCGTTTTGTCGTT) (SEQ ID NO: 4) were synthesized with nuclease-resistant phosphorothioate backbones by Hybridon (Milford, Mass.). LPS level in ODN was undetectable (<1 ng/mg) by Limulus assay (Whittaker Bioproducts, Walkersville, Md.). Cholera toxin (CT) was obtained from Sigma (St. Louis, Mo.).

Mucosal immunization of mice: Each animal was immunized with HBsAg (10 or 100 μg), TT (10 or 100 μg), FLUVIRAL® (50 μl, equivalent to 1/10 human dose, contains 1.5 μg A/Sydney/5/97 HA, 1.5 μg A/Beijing/262/95 HA, 1.5 μg B/Harbin/7/94 HA), either alone or in combination with 10, 100 or 500 μg of ODN (CpG or non-CpG) or with 1 or 10 μg CT. Other groups were immunized with a combination vaccine consisting of 10 μg HBsAg, 10 μg TT and 50 μl FLUVIRAL® with or without the aforementioned adjuvants. For oral immunization, the antigen and adjuvant were made up to a total volume of 50-100 μl with 0.15 M NaCl, and were administered by oral feeding using a 1 c.c. tuberculin syringe (Becton Dickinson, Franklin Lakes, N.J.) attached to a 20-gauge olive tip steel feeding tube (Fine Science Tools Inc., North Vancouver, BC), which was passed through the oral cavity and into the esophagus. For intranasal (IN) immunization, the antigen and adjuvant were made up to a total volume of 5-20 μl with 0.15 M NaCl, which was applied as droplets over both external nares of mice. For intrarectal (IR) immunization, the antigen and adjuvant were made up to a total volume of 20 μl with 0.15 M NaCl and instilled via the anus using a 200 μl pipette tip.

Intramuscular immunization: Each mouse received a single intramuscular (IM) injection with a 0.3 ml insulin syringe (Becton Dickenson, Franklin Lakes, N.J.) into the left tibialis anterior (TA) muscle of 1 μg HBsAg (ay subtype, Medix Biotech, Foster City, Calif.) or 50 μl FLUARIX® (equivalent to 1/10 human dose, contains 1.5 μg A/Sydney/5/97 HA, 1.5 μg A/Beijing/262/95 HA, 1.5 μg B/Harbin/7/94 HA), without or with 10 or 50 μg adjuvant (non-CpG ODN #1982, CpG ODNs #1826, #2006), made up to a total volume of 60 μl with 0.15 M NaCl.

Collection of plasma: Plasma was recovered from mice at various times after immunization by retro-orbital bleeding and stored at −20° C. until assayed.

Collection of mucosal samples: Lung washes were carried out on mice 1 wk after third and final immunization. A 0.33 cc Insulin syringe with a 29G½ needle attached (Becton Dickenson, Franklin Lakes, N.J.) was used for carrying out lung washes. One ml PBS was drawn into the syringe and a length of polyethylene (PE) tubing that was 1 cm longer than the needle was attached (PE20, ID=0.38 mm, Becton Dickinson). The mouse was killed by anesthetic overdose and the trachea was immediately exposed through an anterior midline incision made using fine-tipped surgical scissors (Fine Science Tools Inc., North Vancouver, BC). A small incision was then made in the trachea and a clamp (Fine Science Tools Inc., North Vancouver, BC) was placed above it. The PE tubing was passed a few mm down the trachea through the incision and a second clamp was placed just below the incision to hold the PE tubing in place in the trachea. The PBS solution was slowly instilled in the lungs then withdrawn three times (80% recovery expected). Recovered samples were centrifuge at 13,000 rpm for 7 min., and the supernatants were collected and stored at −20° C. until assayed by ELISA. Vaginal secretion samples were collected by washing the vaginal cavity three times with 75 μl (225 μl total) of PBS containing 0.1 μg sodium azide (Sigma, St. Louis, Mo.). Saliva was obtained following i.p. injection with 100 μl of 1 mg/ml pilocarpine (Sigma) in PBS to induce saliva flow.

Evaluation of immune responses

Systemic humoral response: Antigen-specific antibodies in the mouse plasma were detected and quantified by end-point dilution ELISA assay (in triplicate) for individual animals as described previously (Davis et al., 1998). Briefly, 96-well polystyrene plates (Corning) coated overnight (RT) with HBsAg particles or TT (as used for immunization) (100 μl of 1 or 10 μg/ml for HBsAg and TT respectively, in 0.05 M sodium carbonate-bicarbonate buffer, pH 9.6) were incubated with the plasma for 1 hr at 37° C. Captured antibodies were then detected with horseradish peroxidase (HRP)-conjugated goat anti-mouse IgG, IgG1, IgG2a or IgA (1:4000 in PBS-Tween, 10% FCS: 100 μl/well; Southern Biotechnology Inc., Birmingham, Ala.), followed by addition of o-phenylenediamine dihydrochloride solution (OPD, Sigma), 100 μl/well, for 30 min at RT in the dark. The reaction was stopped by the addition of 4 N H₂SO₄, 50 μl/well. For FLUVIRAL®- and FLUARIX®-specific ELISA assays, coating buffer was PBS, and all dilutions subsequent carried in PBS-Tween, 5% FCS. ). Each bar represents the group geometric mean (±SEM) of the ELISA end-point dilution titer for the specified antibodies in plasma taken 1-4 weeks after final immunization. Titers were defined as the highest plasma dilution (or saliva, vaginal or lung dilution) resulting in an absorbance value two times that of non-immune plasma (or saliva, vaginal or lung), with a cut-off value of 0.05.

Mucosal immune responses: This was carried out on recovered saliva or vaginal or lung washes as for plasma (above) except samples were incubated on coated plates for 2 hr at 37° C. and captured antibodies were detected with HRP-conjugated goat anti-mouse IgA (1:1000 in PBS-Tween. 10% PBS: 100 μl/well; Southern Biotechnology Inc). Non-immune saliva, vaginal or lung wash solutions were used to determine negative control values. End-point dilution titers for IgG in plasma and IgA in mucosal samples were defined as the highest sample dilution that resulted in an absorbance value (OD 450) two times greater than that of non-immune, with a cut-off value of 0.05. Antigen-specific Ig titers were shown for individual animals, or in some cases for a group of animals were expressed as geometric mean titers±the standard error of the mean (GMT±SEM) of individual animal values, which were themselves the average of triplicate assays.

Statistical analysis:

Data were analyzed using the GraphPAD InStat program (GraphPAD Software, San Diego). The statistical significance of the difference between group means was calculated with transformed data (log₁₀) for ELISA titers by Student's 2-tailed t-test for two groups, or by 1-factor analysis of variance (ANOVA) followed by Tukey's test for three or more groups. Differences were considered to be not significant with p>0.05.

RESULTS

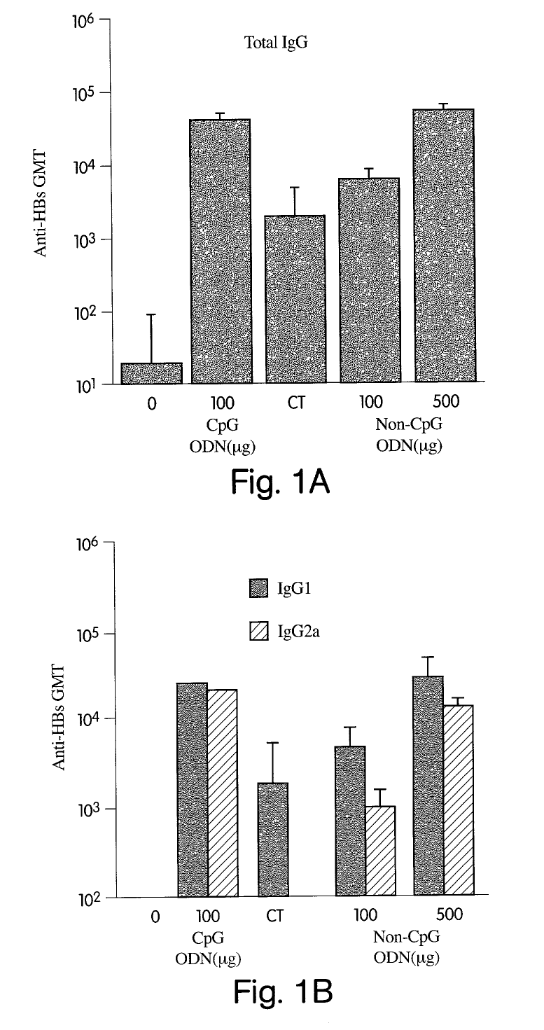

In FIG. 1 mice were immunized by oral delivery with HBsAg (100 μg) without adjuvant or in combination with CpG ODN (motif #1826, 100 μg), non-CpG ODN (motif #1982, 100 or 500 μg) or Cholera toxin (CT, 10 μg). Each bar represents the group geometric mean (±SEM) of the ELISA end-point dilution titer for HBsAg-specific antibodies (anti-HBs GMT) (Total IgG (FIG. 1a) IgG1 (black bars FIG. 1b) or IgG2a (hatched bars FIG. 1b)) in plasma taken 1 week after final immunization.

Oral delivery of HBsAg without adjuvant resulted in none or only low anti-HBs IgG titers in the plasma of mice (FIG. 1a). In contrast, much higher levels of anti-HBs IgG antibodies were detected when CpG ODN #1826 (100 μg), CT (10 μg) or non-CpG ODN #1982 (100 or 500 μg) were added (p<0.05). Compared to results obtained with CT (10 μg), a classical mucosal adjuvant, HBsAg-specific IgG titers with 100 or 500 μg non-CpG ODN were better (100 μg non-CpG ODN, p<0.05) or equally good (500 μg non-CpG ODN, p>0.05). Surprisingly, there was no significant difference between results obtained with an equivalent dose (100 μg) of non-CpG and CpG ODN (p>0.05). When antibody isotypes were used as an indication of the Th-bias of the responses induced by the different formulations, the addition of non-CpG ODN augmented both IgG1 (Th2-like) and IgG2a (Th1-like) but with a predominance of IgG1 (FIG. 1b), as did CT. In contrast, CpG ODN induced an equally mixed Th1/Th2 response, which is much more Th1-biased than is obtained with HBsAg alone (by other routes, where it is effective on its own).

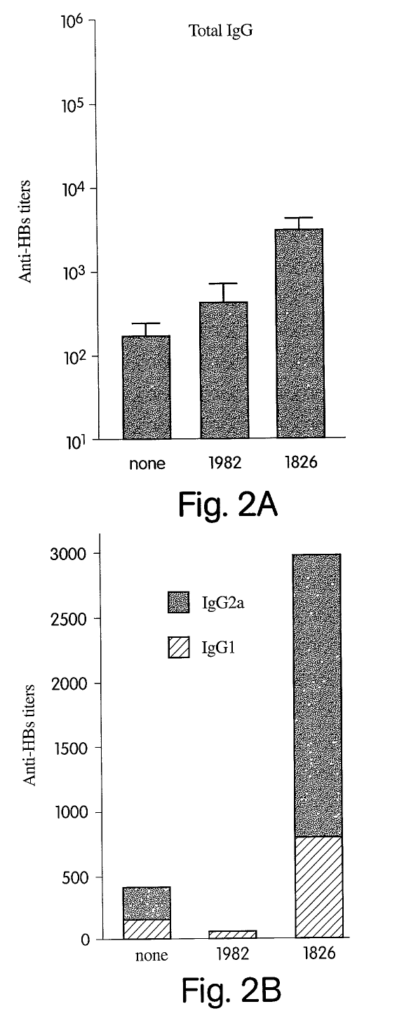

Our findings that oral delivery of HBsAg resulted in enhanced IgG levels with both CpG and non-CpG ODN were particularly surprising since we had previously demonstrated, with IM delivery, an enhancement of immune responses with CpG ODN but not non-CpG ODN (FIG. 2) (Davis et al., 1998). In FIG. 2 mice were immunized by intramuscular (IM) injection with 1 μg HBsAg without adjuvant or with 10 μg of CpG ODN (motif #1826) or non-CpG ODN (motif #1982). Each bar represents the group mean (±SEM) of the ELISA end-point dilution titer for HBsAg-specific antibodies (anti-HBs) (total (FIG. 2a) or IgG1 (hatched bars FIG. 2b) or IgG2a (grey bars FIG. 2b)) in plasma taken 4 weeks after immunization.

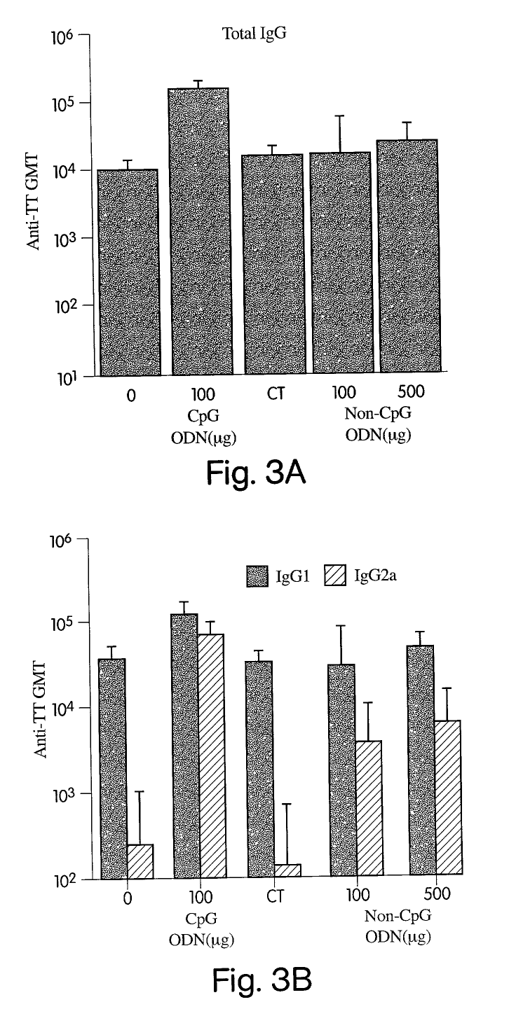

When TT was used as antigen for oral delivery, TT-specific total IgG titers in plasma were similarly increased with both CpG ODN and non-CpG ODN, as long as a low enough dose of TT was used. In FIG. 3 mice were immunized by oral delivery on days 0, 7 and 14 with TT (100 μg) without adjuvant or in combination with CpG ODN (motif #1826, 100 μg), non-CpG ODN (motif #1982, 100 or 500 μg) or Cholera toxin (CT, 10 μg). Each bar represents the group geometric mean (±SEM) of the ELISA end-point dilution titer for TT-specific antibodies (anti-TT GMT) (Total IgG (FIG. 3a)IgG1 (black bars FIG. 3b) or IgG2a (hatched bars FIG. 3b)) in plasma taken 1 week after final immunization.

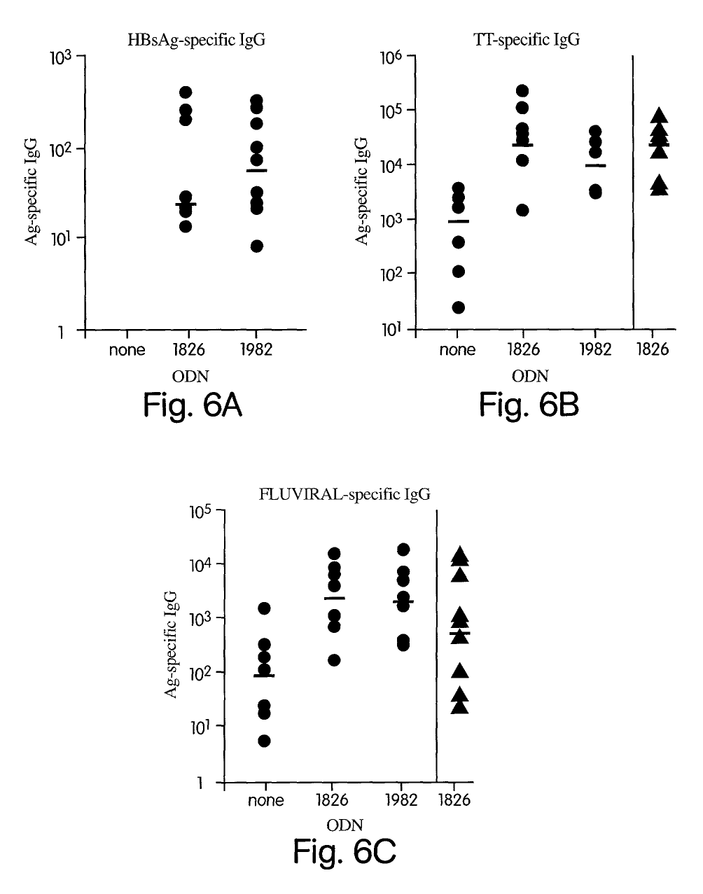

Thus while an effect for CpG ODN but not non-CpG ODN was seen with a very high 100 μg dose of TT (FIG. 3a), both ODN were effective with a 10 μg dose (see FIGS. 6, 8 and 10). Regardless of TT dose however, antibody isotypes indicated that CpG ODN overcame the strong Th2-bias of the antigen, whereas, responses with both non-CpG ODN or CT remained Th2 (IgG1>>IgG2a) (FIG. 3b).

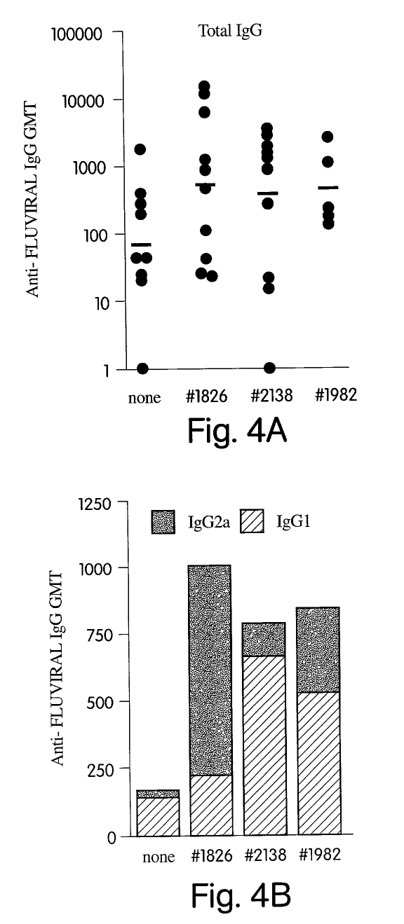

FLUVIRAL® was used as antigen for oral delivery in FIG. 4. In FIG. 4 mice were immunized by oral delivery on days 0, 7 and 14 with FLUVIRAL® (50 μl, 1/10 human dose) without adjuvant or in combination with 10 μg of CpG ODN (motif #1826) or non-CpG ODN (motif #2138 or #1982). Each bar represents the group geometric mean (±SEM) of the ELISA end-point dilution titer for FLUVIRAL®-specific antibodies (anti-FLUVIRAL® GMT) (Total IgG (FIG. 4a) IgG1 (hatched bars FIG. 4b) or IgG2a (black bars FIG. 4b)) in plasma taken 1 week after final immunization. When FLUVIRAL® was used as antigen for oral delivery, mean FLUVIRAL®-specific IgG titers in plasma were augmented similarly (approximately 5-fold) with both non-CpG ODNs (#2138 and #1982) and CpG ODN (#1826) (FIG. 4a). However, whereas the addition of CpG ODN augmented predominantly IgG2a (Th-1 like) antibodies and therefore overcame the strong Th-2 bias of FLUVIRAL® alone, the non-CpG ODN augmented both IgG1 and IgG2a such that the Th2 bias was retained (FIG. 4b).

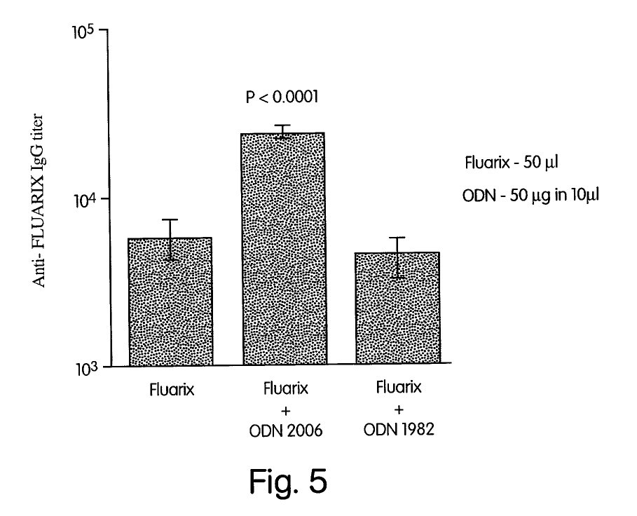

Similar to our findings with HBsAg (FIG. 2), when a similar influenza virus vaccine (FLUARIX®) was administered IM, no augmentation of Antigen-specific IgG was seen with non-CpG ODN (FIG. 5), indicating that the immunostimulatory properties of non-CpG ODN are associated with mucosal but not parenteral delivery, at least at low concentrations. In FIG. 5 mice were immunized by intramuscular (IM) injection with FLUARIX® (50 μl, 1/10 human dose) without adjuvant or in combination with 50 μg of CpG ODN (motif #2006) or non-CpG ODN (motif#1982). Each bar represents the group mean (±SEM) of the ELISA end-point dilution titer for FLUARIX®-specific antibodies (anti-FLUARIX®) in plasma taken 2 weeks after immunization.

In order to determine whether similar effects would be seen with a multivalent vaccine, mice were immunized orally with a combination of HBsAg/TT/FLUVIRAL® alone or with CpG (#1826) or non-CpG (#1982) ODN. In FIG. 6 mice were immunized by oral delivery on days 0, 7 and 14 with a combination of HBsAg/TT/FLUVIRAL® (10 μg, 10 μg, 50 μl respectively) without adjuvant or in combination with 10 μg CpG ODN (motif #1826), or non-CpG ODN (motif #1982). Each symbol represents the ELISA end-point dilution titer for HBsAg-specific (FIG. 6a), TT-specific (FIG. 6b), or FLUVIRAL®-specific (FIG. 6c) antibodies in plasma of individual mice taken 1 week after final immunization with multiple antigens (HBsAg/TT/FLUVIRAL®, filled circles) or with a single antigen (TT (FIG. 6b) or FLUVIRAL® (FIG. 6c), filled triangles). Horizontal bars represent the group geometric mean.

Oral delivery of HBsAg/TT/FLUVIRAL® without adjuvant resulted in no detectable HBsAg-specific IgG in the plasma of mice and mean TT- and FLUVIRAL®-specific IgG titers were ˜1000 and 100 respectively (FIG. 6). In contrast, when CpG or non-CpG ODN was added mean TT- and FLUVIRAL®-specific IgG titers were raised ˜10- to 20-fold and HBsAg-specific IgG was now detected. The combination of different antigens did not result in any competitive inhibition since Antigen-specific titers attained with multiple antigens were as high as those attained with single antigens (FIG. 6b and c, triangle symbols).

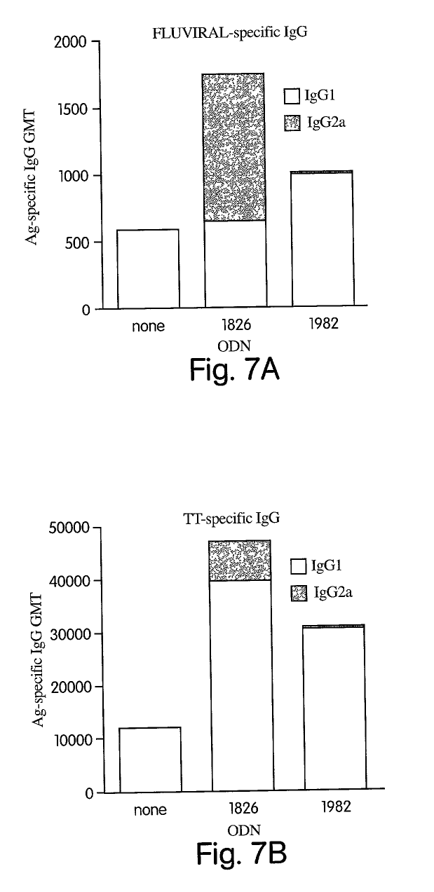

As we had seen with single antigens, the addition of CpG ODN enhanced Th1-like responses (IgG2a>>IgG1), whereas with non-CpG, Th2-like responses were enhanced (IgG1>>IgG2a) (FIG. 7). In FIG. 7 mice were immunized by oral delivery on days 0, 7 and 14 with a combination of HBsAg/TT/FLUVIRAL® (10 μg, 10 μg, 50 μl respectively) without adjuvant or in combination with 10 μg CpG ODN (motif # 1826), or non-CpG ODN (motif #1982). Each bar represents the group geometric mean of the ELISA end-point dilution titer for FLUVIRAL®-specific (FIG. 7a) or TT-specific (FIG. 7b) antibodies of IgG1 (grey bars) or IgG2a (black bars) isotypes in plasma taken 1 week after final immunization. Titers were defined as the highest plasma dilution resulting in an absorbance value two times that of non-immune plasma, with a cut-off value of 0.05.

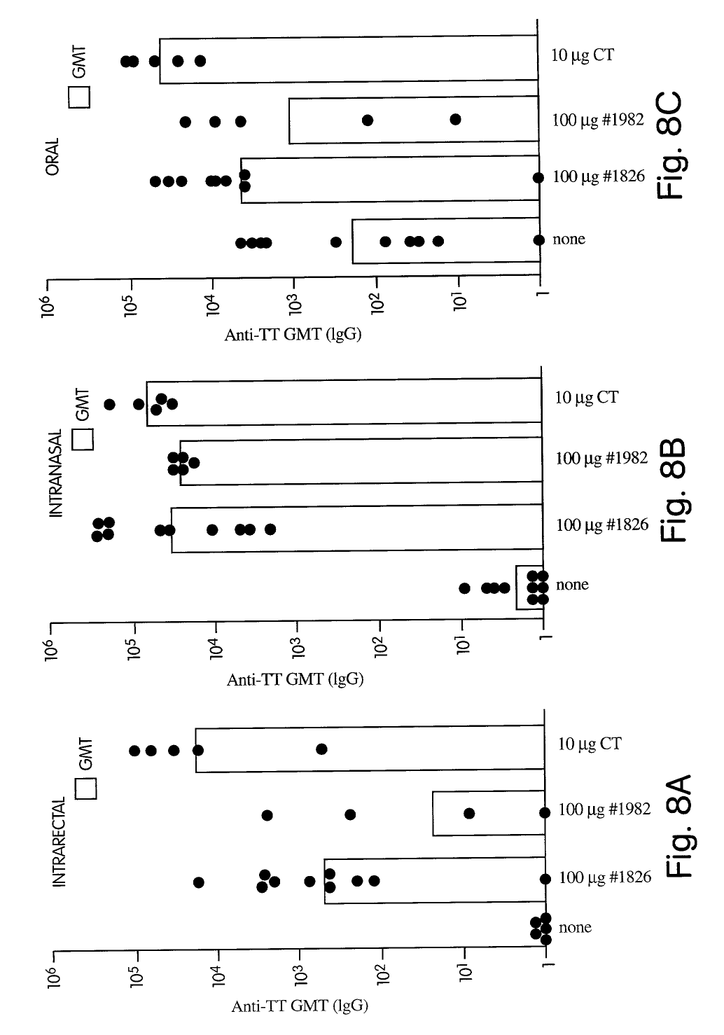

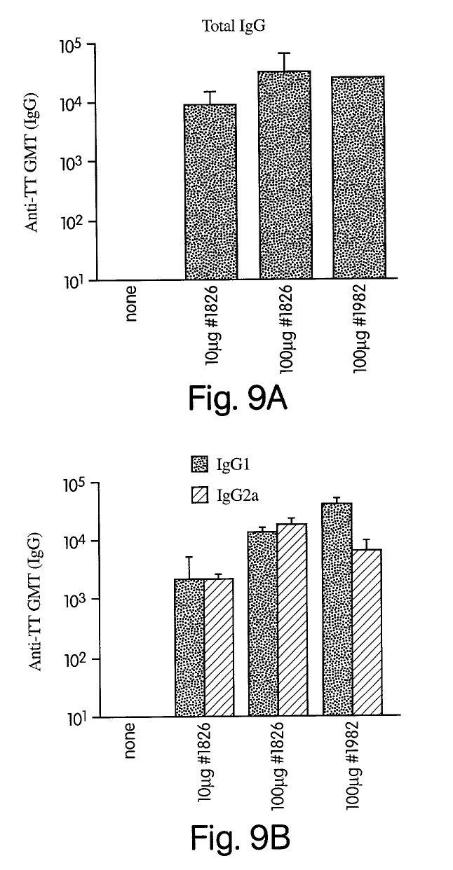

In order to determine whether non-CpG ODN would also have stimulatory effects when delivered by different mucosal routes, mice were immunized with TT (10 μg) either alone, or with CpG or non-CpG ODN (100 μg) as adjuvant by intrarectal (IR, FIG. 8a), intranasal (IN, FIG. 8b and FIG. 9) as well as oral routes (FIG. 8c). In addition, control mice were immunized using CT, a conventional mucosal adjuvant (FIG. 8). In FIG. 8 CpG ODN (motif#1826, 100 μg), non-CpG ODN (motif#1982, 100 μg) or Cholera toxin (CT, 10 μg) were used as adjuvant and in FIG. 9 with CpG ODN (motif #1826, 10 or 100 μg) or non-CpG ODN (motif #1982, 100 μg) were used as adjuvant. Each filled circle in FIG. 8 represents the ELISA end-point dilution titer for TT-specific antibodies in plasma of individual mice taken 1 week after final immunization. Grey bars represent the group geometric mean. Each bar in FIG. 9 represents the group geometric mean (±SEM) of the ELISA end-point dilution titer for TT-specific antibodies (anti-TT GMT) of Total IgG (FIG. 9a) or IgG1 (grey bars) or IgG2a (hatched bars) isotypes (FIG. 9b) in plasma taken 1 week after final immunization.

Non-CpG ODN was found to have a stimulatory effect when delivered by all mucosal routes tested. Delivery of TT by the IR route resulted in 0/5, 8/10, ⅖ and 5/5 mice responding (anti-TT IgG in plasma>100) for no adjuvant, CpG ODN, non-CpG ODN and CT respectively; by the IN route resulted in 0/10, 10/10, 5/5 and 5/5 mice responding for no adjuvant, CpG ODN, non-CpG ODN and CT respectively; and for oral delivery resulted in 5/10, 8/9, ⅘ and 5/5 mice responding for no adjuvant, CpG ODN, non-CpG ODN and CT respectively (FIG. 8). Similar to our findings with oral delivery, when non-CpG ODN were administered by IN delivery an equivalent response was induced to that with CpG ODN or CT (p<0.05) (FIG. 8 and FIG. 9a), however, the response with non-CpG ODN was more Th2-like (IgG1>IgG2a) than with CpG ODN (IgG1=IgG2a) (FIG. 9b).

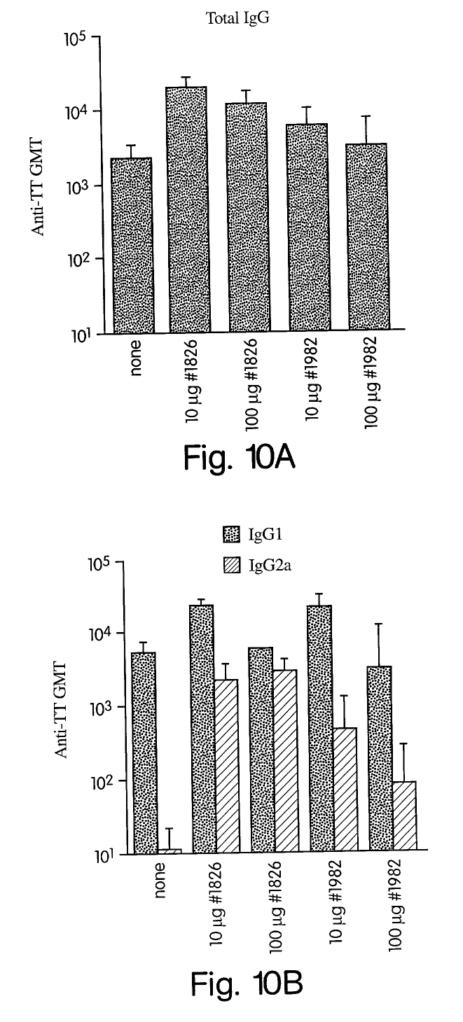

In FIG. 10 mice were immunized by oral delivery on days 0, 7 and 14 with TT (10 μg) without adjuvant or in combination with CpG ODN (motif #1826, 10 or 100 μg) or non-CpG ODN (motif #1982, 10 or 100 μg). Each bar represents the group geometric mean SEM) of the ELISA end-point dilution titer for TT-specific antibodies (anti-TT GMT) of Total (FIG. 10a) or IgG1 (grey bars) or IgG2a (hatched bars) isotypes (FIG. 10b) in plasma taken 1 week after final immunization. The immunostimulatory effects of non-CpG ODN after oral delivery were observed at both low (10 □g) and high (100 μg) doses of non-CpG ODN (FIG. 10a), and, in contrast to CpG DNA, increasing the dose of non-CpG ODN did not alter the IgG2a to IgG1 ratio (FIG. 10b).

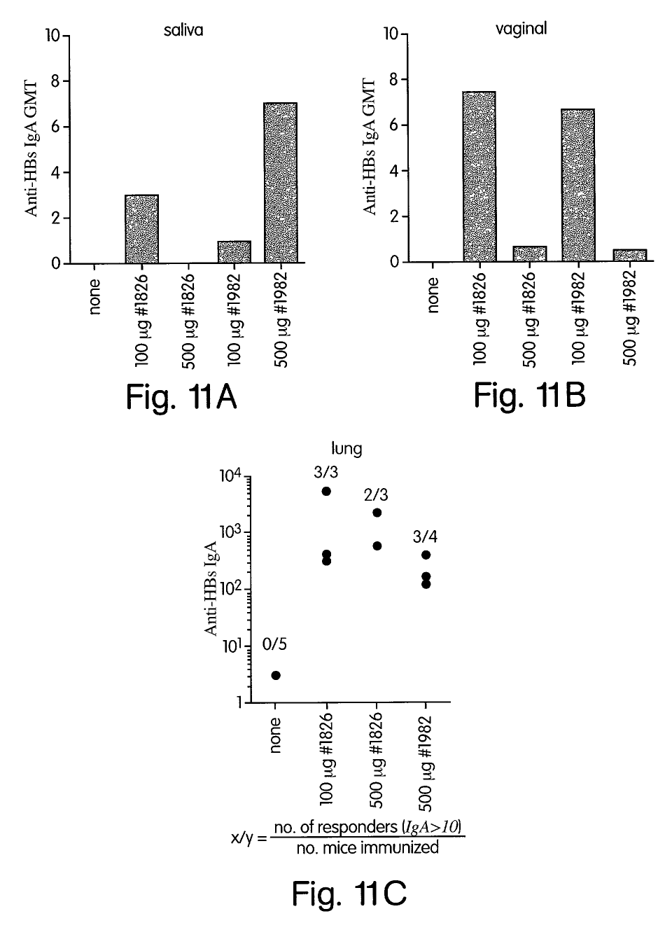

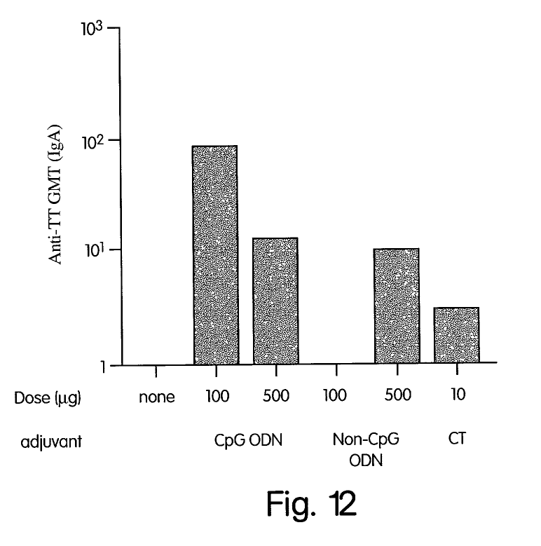

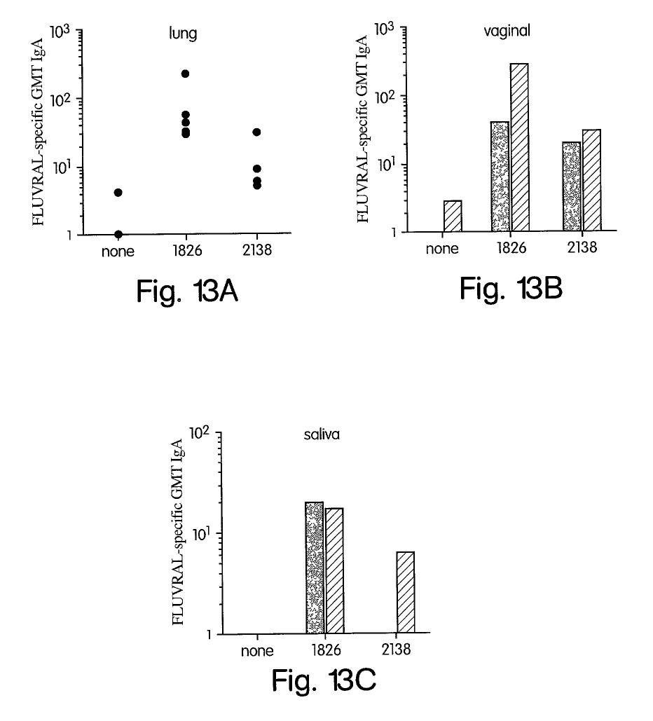

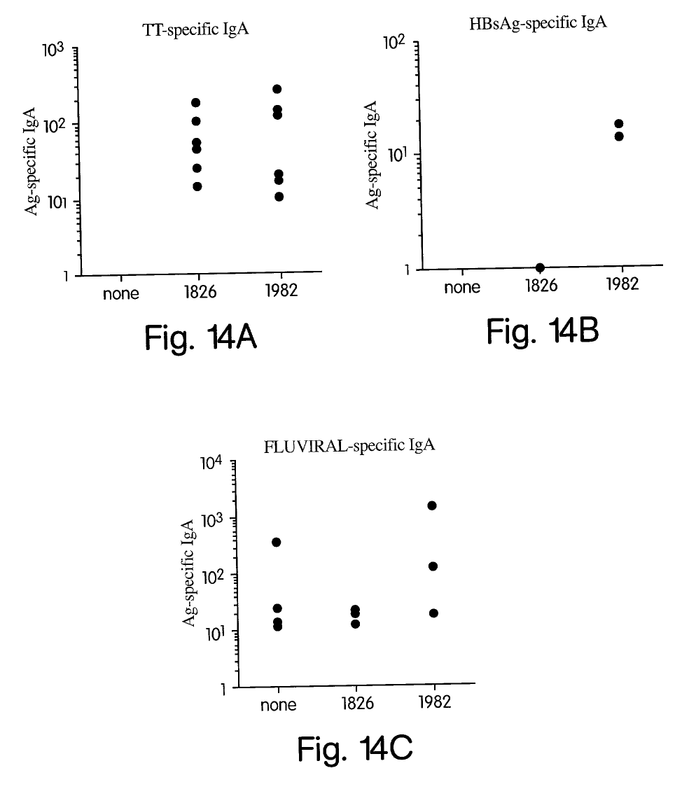

In addition to augmenting systemic immune responses (IgG), non-CpG ODN was also found to augment antigen-specific mucosal immunity (IgA) at a number of mucosal sites. This was found with administration of single antigens, namely HBsAg (FIG. 11), TT (FIG. 12), and FLUVIRAL® (FIG. 13), or multiple antigens, namely HBsAg/TT/FLUVIRAL® (FIG. 14). These findings are important since secretory IgA is thought to protect against pathogen entry to the body via a mucosal surface.

In FIG. 11 mice were immunized by oral delivery on days 0, 7 and 14 with HBsAg (100 μg) without adjuvant or in combination with CpG ODN (motif #1826, 100 or 500 μg), or non-CpG ODN (motif #1982, 100 or 500 μg). Each bar represents the ELISA end-point dilution titer for HBsAg-specific IgA antibodies (anti-HBs IgA) in saliva (FIG. 11a), vaginal washes (FIG. 11b), or lung washes (FIG. 11c) taken 1 week after final immunization and pooled for each group.

Mice were immunized, in FIG. 12, by oral delivery on days 0, 7 and 14 with TT (100 μg) without adjuvant or in combination with CpG ODN (motif #1826, 100 or 500 μg), non-CpG ODN (motif #1982, 100 or 500 μg) or Cholera toxin (CT, 10 μg). Each bar represents the ELISA end-point dilution titer for TT-specific IgA antibodies (anti-TT IgA) in vaginal washes collected 1 week after final immunization and pooled for each group.

In FIG. 13 mice were immunized by oral delivery on days 0, 7 and 14 with FLUVIRAL® (50 μl, 1/10 human dose) without adjuvant or in combination with 10 μg of CpG ODN (motif#1826) or non-CpG ODN (motif #2138). Each filled circle represents the ELISA end-point dilution titer for FLUVIRAL®-specific IgA antibodies (anti-FLUVIRAL® IgA) for individual mice in lung washes (FIG. 13a), vaginal washes (FIG. 13b), or saliva (FIG. 13c) taken 1 week after final immunization. Grey and black bars in FIGS. 13b and 13c represent identical treatments given to two separate groups of animals.

In FIG. 14 mice were immunized by oral delivery on days 0, 7 and 14 with a combination of HBsAg/TT/FLUVIRAL® (10 μg, 10 μg, 50 μl respectively) without adjuvant or in combination with 10 μg CpG ODN (motif #1826), or non-CpG ODN (motif #1982). Each symbol represents the ELISA end-point dilution titer for HBsAg-specific IgA (FIG. 14b), TT-specific (FIG. 14a), or FLUVIRAL®-specific (FIG. 14c) antibodies in lung washes of individual mice taken 1 week after final immunization.

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

We claim:

1. A method for inducing an antigen specific response comprising:
administering to a subject an antigen and a Th2-immunostimulatory nucleic acid in an amount effective to produce an antigen specific immune response when the Th2-immunostimulatory nucleic acid is administered mucosally or dermally.

2. The method of claim 1, wherein the subject is administered the antigen after the Th2-immunostimulatory nucleic acid.

3. The method of claim 1, wherein the subject is administered the antigen before the Th2-immunostimulatory nucleic acid.

4. The method of claim 1, wherein the subject is administered the antigen and the Th2-immunostimulatory nucleic acid simultaneously.

5. The method of claim 1, wherein the Th2-immunostimulatory nucleic acid is delivered to the mouth, skin or eye.

6. The method of claim 1, further comprising administering a therapeutic agent to the subject.

7. The method of claim 6, wherein the therapeutic agent is a Th1 adjuvant.

8. The method of claim 7, wherein the Th1 adjuvant is selected from the group consisting of CpG nucleic acids, MF59, SAF, MPL, and QS21.

9. The method of claim 7, wherein the Th1 adjuvant is administered following the administration of the Th2-immunostimulatory nucleic acid.

10. The method of claim 6, wherein the therapeutic agent is a Th2 adjuvant.

11. The method of claim 10, wherein the Th2 adjuvant is selected from the group consisting of adjuvants that create a depot effect, adjuvants that stimulate the immune system, and adjuvants that create a depot effect and stimulate the immune system and mucosal adjuvants.

12. The method of claim 11, wherein the adjuvant that creates a depot effect is selected from the group consisting of alum; emulsion-based formulations including mineral oil, non-mineral oil, water-in-oil or oil-in-water-in oil emulsion, oil-in-water emulsions such as Seppic ISA series of Montanide adjuvants; and PROVAX.

13. The method of claim 11, wherein the adjuvant that stimulates the immune system is selected from the group consisting of saponins purified from the bark of the Q. saponaria tree; poly[di(carboxylatophenoxy)phosphazene; derivatives of lipopolysaccharides, muramyl dipeptide and threonyl-muramyl dipeptide; OM-174; and Leishmania elongation factor.

14. The method of claim 11, wherein the adjuvant that creates a depot effect and stimulates the immune system is selected from the group consisting of ISCOMs; SB-AS2; SB-AS4; non-ionic block copolymers that form micelles such as CRL 1005; and Syntex Adjuvant Formulation.

15. The method of claim 11, wherein the mucosal adjuvant is selected from the group consisting of CpG nucleic acids, Bacterial toxins, Cholera toxin, CT derivatives, CT B subunit; CTD53; CTK97; CTK104; CTD53/K63; CTH54; CTN107; CTE114; CTE112K; CTS61F; CTS106; and CTK63, Zonula occludens toxin, zot, Escherichia coli heat-labile enterotoxin, Labile Toxin, LT derivatives, LT B subunit; LT7K; LT61F; LT112K; LT118E; LT146E; LT192G; LTK63; and LTR72, Pertussis toxin, PT-9K/129G; Toxin derivatives; Lipid A derivatives, MDP derivatives; Bacterial outer membrane proteins, outer surface protein A (OspA) lipoprotein of Borrelia burgdorferi, outer membrane protein of Neisseria meningitidis; Oil-in-water emulsions, Aluminum salts; and Saponins, ISCOMs, the Seppic ISA series of Montanide adjuvants, Montanide ISA 720; PROVAX; Syntext Adjuvant Formulation; poly[di(carboxylatophenoxy) phosphazene and Leishmania elongation factor.

16. The method of claim 6, wherein the therapeutic agent is a cytokine.

17. The method of claim 1, wherein the Th2-immunostimulatory nucleic acid is formulated in a form selected from the group consisting of a liquid solution, a powder, a microparticle, and a bioadhesive polymer.

18. The method of claim 1, wherein the Th2-immunostimulatory nucleic acid is administered by a route selected from the group consisting of oral, intranasal, vaginal, rectal, intra-ocular, and by inhalation.

19. The method of claim 1, wherein the Th2-immunostimulatory nucleic acid is administered by a route selected from the group consisting of intradermal, intraepidermal and transdermal.

20. The method of claim 1, wherein the antigen specific immune response is a systemic immune response.

21. The method of claim 1, wherein the antigen specific immune response is a mucosal immune response.

22. The method of claim 1, wherein the Th2-immunostimulatory nucleic acid is administered using a delivery system selected from the group consisting of a needleless delivery system, a scarification delivery system, and a tyne delivery system.

23. The method of claim 1, wherein the antigen is administered using a delivery system selected from the group consisting of a needleless delivery system, a scarification delivery system, and a tyne delivery system.

24. The method of claim 6, wherein the therapeutic agent is selected from the group consisting of an anti-viral agent, an anti-bacterial agent, an anti-parasitic agent, an anti-fungal agent, and cancer medicament.

25. The method of claim 1, wherein the antigen is selected from the group of antigens consisting of viral antigens, fungal antigens, bacterial antigens, parasitic antigens, and cancer antigens.

26. The method of claim 1, wherein the subject has not been exposed to an Th1 immunostimulatory nucleic acid prior to administration of the Th2 immunostimulatory nucleic acid.

27. The method of claim 1, wherein the subject is not experiencing a Th1 mediated disorder at the time of administration.

28. The method of claim 1, wherein the antigen is not conjugated to the Th2 immunostimulatory nucleic acid.

29. The method of claim 1, wherein the antigen is not a self antigen.

30. The method of claim 1, wherein the antigen is not an extracellular antigen.

31. A method for inducing an antigen specific response comprising:
administering to a subject an antigen and a Th2-immunostimulatory nucleic acid in an amount effective to produce an antigen specific immune response when the Th2-immunostimulatory nucleic acid is administered parenterally.

32. The method of claim 31, wherein the subject is administered the antigen after the Th2-immunostimulatory nucleic acid.

33. The method of claim 31, wherein the subject is administered the antigen before the Th2-immunostimulatory nucleic acid.

34. The method of claim 31, wherein the subject is administered the antigen and the Th2-immunostimulatory nucleic acid simultaneously.

35. The method of claim 31, wherein the Th2-immunostimulatory nucleic acid is delivered intravenously, intraperitoneally, intramuscularly, subcutaneously, or by infusion.

36. The method of claim 31, further comprising administering a therapeutic agent to the subject.

37. The method of claim 36, wherein the therapeutic agent is a Th1 adjuvant.

38. The method of claim 37, wherein the Th1 adjuvant is selected from the group consisting of CpG nucleic acids, MF59, SAF, MPL, and QS21.

39. The method of claim 36, wherein the therapeutic agent is a Th2 adjuvant.

40. The method of claim 39, wherein the Th2 adjuvant is selected from the group consisting of adjuvants that creates a depot effect, adjuvants that stimulate the immune system, adjuvants that create a depot effect and stimulate the immune system and mucosal adjuvants.

41. The method of claim 40, wherein the adjuvant that creates a depot effect is selected from the group consisting of alum; emulsion-based formulations including mineral oil, non-mineral oil, water-in-oil or oil-in-water-in oil emulsion, oil-in-water emulsions such as Seppic ISA series of Montanide adjuvants; and PROVAX.

42. The method of claim 40, wherein the adjuvant that stimulates the immune system is selected from the group consisting of saponins purified from the bark of the Q. saponaria tree; poly[di(carboxylatophenoxy)phosphazene; derivatives of lipopolysaccharides, muramyl dipeptide and threonyl-muramyl dipeptide; OM-174; and Leishmania elongation factor.

43. The method of claim 40, wherein the adjuvant that creates a depot effect and stimulates the immune system is selected from the group consisting of ISCOMs; SB-AS2; SB-AS4; non-ionic block copolymers that form micelles such as CRL 1005; and Syntex Adjuvant Formulation.

44. The method of claim 40, wherein the mucosal adjuvant is selected from the group consisting of CpG nucleic acids, Bacterial toxins, Cholera toxin, CT derivatives, CT B subunit; CTD53; CTK97; CTK104; CTD53/K63; CTH54; CTN107; CTE114; CTE112K; CTS61F; CTS106; and CTK63, Zonula occludens toxin, zot, Escherichia coli heat-labile enterotoxin, Labile Toxin, LT derivatives, LT B subunit; LT7K; LT61F; LT112K; LT118E; LT146E; LT192G; LTK63; and LTR72, Pertussis toxin, PT-9K/129G; Toxin derivatives; Lipid A derivatives, MDP derivatives; Bacterial outer membrane proteins, outer surface protein A (OspA) lipoprotein of Borrelia burgdorferi, outer membrane protein of Neisseria meningitidis; Oil-in-water emulsions, Aluminum salts; and Saponins, ISCOMs, the Seppic ISA series of Montanide adjuvants, Montanide ISA 720; PROVAX; Syntext Adjuvant Formulation; poly[di(carboxylatophenoxy) phosphazene and Leishmania elongation factor.

45. The method of claim 36, wherein the therapeutic agent is a cytokine.

46. The method of claim 31, wherein the Th2-immunostimulatory nucleic acid is formulated in a form selected from the group consisting of a liquid solution, a powder, a microparticle, and a bioadhesive polymer.

47. The method of claim 31, wherein the antigen is a non-extracellular antigen.

48. The method of claim 31, wherein the antigen specific immune response is a systemic immune response.

49. The method of claim 31, wherein the antigen is administered using a delivery system selected from the group consisting of a needleless delivery system, a scarification delivery system, and a tyne delivery system.

50. The method of claim 36, wherein the therapeutic agent is selected from the group consisting of an anti-viral agent, an anti-bacterial agent, an anti-parasitic agent, an anti-fungal agent, and cancer medicament.

51. The method of claim 31, wherein the antigen is selected from the group of antigens consisting of viral antigens, fungal antigens, yeast antigens, parasitic antigens, and tumor (i.e., cancer) antigens.

52. The method of claim 31, wherein the subject has not been exposed to an Th1 immunostimulatory nucleic acid prior to administration of the Th2 immunostimulatory nucleic acid.

53. The method of claim 31, wherein the antigen is not conjugated to the Th2 immunostimulatory nucleic acid.

54. The method of claim 31, wherein the antigen is not a self antigen.

55. A method for treating a non-autoimmune Th1-mediated disease, comprising:
administering to a subject a Th2 immunostimulatory nucleic acid in an amount effective to produce a Th2 immune response when administered mucosally or dermally.

56. The method of claim 55, wherein an antigen is not administered to the subject.

57. The method of claim 55, wherein the subject has not been exposed to a Th1 immunostimulatory nucleic acid.

58. The method of claim 55, wherein the non-autoimmune Th1-mediated disease is not mediated by a Th1 immunostimulatory nucleic acid.

59. The method of claim 56, wherein the disorder is selected from the group consisting of psoriasis, Th1 inflammatory disorders, solid organ allograft rejection, symptoms associated with Hepatitis B infection, insulin-dependent diabetes mellitus, multiple sclerosis, “Silent thyroiditis”, and unexplained recurrent abortion.

60. The method of claim 55, wherein the method is a method for inducing a local Th2 environment in the subject.

61. The method of claim 60, wherein the local Th2 environment is in the skin and wherein the subject has a Th1 mediated skin disorder.

62. The method of claim 60, wherein the local Th2 environment is in the eye and the subject has a viral infection.

63. The method of claim 62, wherein the viral infection is HSV-1.

64. The method of claim 55, wherein the Th2-immunostimulatory nucleic acid is administered locally.

65. The method of claim 64, wherein the Th2-immunostimulatory nucleic acid is administered to a tissue selected from the group consisting of skin and eye.

66. The method of claim 55, further comprising administering a therapeutic agent to the subject.

67. The method of claim 66, wherein the therapeutic agent is a Th1 adjuvant.

68. The method of claim 67, wherein the Th1 adjuvant is selected from the group consisting of CpG nucleic acids, MF59, SAF, MPL, and QS21.

69. The method of claim 66, wherein the therapeutic agent is a Th2 adjuvant.

70. The method of claim 69, wherein the Th2 adjuvant is selected from the group consisting of adjuvants that creates a depot effect, adjuvants that stimulate the immune system, adjuvants that create a depot effect and stimulate the immune system and mucosal adjuvants.

71. The method of claim 70, wherein the adjuvant that creates a depot effect is selected from the group consisting of alum; emulsion-based formulations including mineral oil, non-mineral oil, water-in-oil or oil-in-water-in oil emulsion, oil-in-water emulsions such as Seppic ISA series of Montanide adjuvants; and PROVAX.

72. The method of claim 70, wherein the adjuvant that stimulates the immune system is selected from the group consisting of saponins purified from the bark of the Q. saponaria tree; poly[di(carboxylatophenoxy)phosphazene; derivatives of lipopolysaccharides, muramyl dipeptide and threonyl-muramyl dipeptide; OM-174; and Leishmania elongation factor.

73. The method of claim 70, wherein the adjuvant that creates a depot effect and stimulates the immune system is selected from the group consisting of ISCOMs; SB-AS2; SB-AS4; non-ionic block copolymers that form micelles such as CRL 1005; and Syntex Adjuvant Formulation.

74. The method of claim 70, wherein the mucosal adjuvant is selected from the group consisting of CpG nucleic acids, Bacterial toxins, Cholera toxin, CT derivatives, CT B subunit; CTD53; CTK97; CTK104; CTD53/K63; CTH54; CTN107; CTE114; CTE112K; CTS61F; CTS106; and CTK63, Zonula occludens toxin, zot, Escherichia coli heat-labile enterotoxin, Labile Toxin, LT derivatives, LT B subunit; LT7K; LT61F; LT112K; LT118E; LT146E; LT192G; LTK63; and LTR72, Pertussis toxin, PT-9K/129G; Toxin derivatives; Lipid A derivatives, MDP derivatives; Bacterial outer membrane proteins, outer surface protein A (OspA) lipoprotein of Borrelia burgdorferi, outer membrane protein of Neisseria meningitidis; Oil-in-water emulsions, Aluminum salts; and Saponins, ISCOMs, the Seppic ISA series of Montanide adjuvants, Montanide ISA 720; PROVAX; Syntext Adjuvant Formulation; poly[di(carboxylatophenoxy) phosphazene and Leishmania elongation factor.

75. The method of claim 66, wherein the therapeutic agent is a cytokine.

76. The method of claim 66, wherein the therapeutic agent is a drug for treating Th1 mediated disorders.

77. The method of claim 76, wherein the drug for treating Th1 mediated disorders is selected from the group consisting of anti-psoriasis creams, eye drops, nose drops, Sulfasalazine, glucocorticoids, propylthiouracil, methimazole, ¹³¹, insulin, IFN-β1a, IFN-β1b, copolymer 1 (i.e., MS), glucocorticoids (i.e., MS), ACTH, avonex, azathioprine, cyclophosphamide, UV-B, PUVA, methotrexate, calcipitriol, cyclophosphamide, OKT3, FK-506, cyclosporin A, azathioprine, and mycophenolate mofetil.

78. A method for treating an autoimmune disease, comprising:
administering to a subject a Th2-immunostimulatory nucleic acid in an amount effective to produce a Th2 immune response when administered mucosally or dermally, wherein the subject has not been exposed to a Th1 immunostimulatory nucleic acid.

79. The method of claim 78, wherein the autoimmune disease is selected from the group consisting of rheumatoid arthritis, Crohn's disease, systemic lupus erythematosus (SLE), autoimmune encephalomyelitis, myasthenia gravis, Hashimoto's thyroiditis, Goodpasture's syndrome, pemphigus, Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis, bullous pemphigoid, Sjögren's syndrome, insulin resistance, and autoimmune diabetes mellitus.

80. The method of claim 78, further comprising administering to the subject a self antigen, to produce an immune hyporesponsive state.

81. The method of claim 80, wherein the self antigen is not conjugated to the Th2 immunostimulatory nucleic acid.

82. The method of claim 78, wherein the method is a method for inducing a local Th2 environment in the subject.

83. The method of claim 82, wherein the local Th2 environment is in the skin.

84. The method of claim 82, wherein the local Th2 environment is in the eye.

85. The method of claim 78, wherein the Th2-immunostimulatory nucleic acid is administered mucosally.

86. The method of claim 78, wherein the Th2-immunostimulatory nucleic acid is administered locally.

87. The method of claim 86, wherein the Th2-immunostimulatory nucleic acid is administered to a tissue selected from the group consisting of skin and eye.

88. The method of claim 78, further comprising administering a therapeutic agent to the subject.

89. The method of claim 88, wherein the therapeutic agent is a Th1 adjuvant.

90. The method of claim 89, wherein the Th1 adjuvant is selected from the group consisting of CpG nucleic acids, MF59, SAF, MPL, and QS21.

91. The method of claim 88, wherein the therapeutic agent is a Th2 adjuvant.

92. The method of claim 91, wherein the Th2 adjuvant is selected from the group consisting of adjuvants that creates a depot effect, adjuvants that stimulate the immune system, adjuvants that create a depot effect and stimulate the immune system and mucosal adjuvants.

93. The method of claim 92, wherein the adjuvant that creates a depot effect is selected from the group consisting of alum; emulsion-based formulations including mineral oil, non-mineral oil, water-in-oil or oil-in-water-in oil emulsion, oil-in-water emulsions such as Seppic ISA series of Montanide adjuvants; and PROVAX.

94. The method of claim 92, wherein the adjuvant that stimulates the immune system is selected from the group consisting of saponins purified from the bark of the Q. saponaria tree; poly[di(carboxylatophenoxy)phosphazene; derivatives of lipopolysaccharides, muramyl dipeptide and threonyl-muramyl dipeptide; OM-174; and Leishmania elongation factor.

95. The method of claim 92, wherein the adjuvant that creates a depot effect and stimulates the immune system is selected from the group consisting of ISCOMs; SB-AS2; SB-AS4; non-ionic block copolymers that form micelles such as CRL 1005; and Syntex Adjuvant Formulation.

96. The method of claim 92, wherein the mucosal adjuvant is selected from the group consisting of CpG nucleic acids, Bacterial toxins, Cholera toxin, CT derivatives, CT B subunit; CTD53; CTK97; CTK104; CTD53/K63; CTH54; CTN107; CTE114; CTE112K; CTS61F; CTS106; and CTK63, Zonula occludens toxin, zot, Escherichia coli heat-labile enterotoxin, Labile Toxin, LT derivatives, LT B subunit; LT7K; LT6 IF; LT112K; LT118E; LT146E; LT192G; LTK63; and LTR72, Pertussis toxin, PT-9K/129G; Toxin derivatives; Lipid A derivatives, MDP derivatives; Bacterial outer membrane proteins, outer surface protein A (OspA) lipoprotein of Borrelia burgdorferi, outer membrane protein of Neisseria meningitidis; Oil-in-water emulsions, Aluminum salts; and Saponins, ISCOMs, the Seppic ISA series of Montanide adjuvants, Montanide ISA 720; PROVAX; Syntext Adjuvant Formulation; poly[di(carboxylatophenoxy) phosphazene and Leishmania elongation factor.

97. The method of claim 88, wherein the therapeutic agent is a cytokine.

98. The method of claim 88, wherein the therapeutic agent is a drug for treating autoimmune disease.

99. The method of claim 98, wherein the drug for treating Th1 mediated disorders is selected from the group consisting of anti-psoriasis creams, eye drops, nose drops, Sulfasalazine, glucocorticoids, propylthiouracil, methimazole, ¹³¹, insulin, IFN-β1a, IFN-β1b, copolymer 1 (i.e., MS), glucocorticoids (i.e., MS), ACTH, avonex, azathioprine, cyclophosphamide, UV-B, PUVA, methotrexate, calcipitriol, cyclophosphamide, OKT3, FK-506, cyclosporin A, azathioprine, and mycophenolate mofetil.

100. A pharmaceutical composition, comprising:
an effective amount of a Th2 immunostimulatory nucleic acid for stimulating a Th2 immune response when administered mucosally or dermally, an antigen, and a pharmaceutically acceptable carrier.

101. The pharmaceutical composition of claim 100, wherein the antigen is not conjugated to the Th2 immunostimulatory nucleic acid.

102. The pharmaceutical composition of claim 100, wherein the Th2 immune response is a mucosal immune response.

103. The pharmaceutical composition of claim 100, wherein the Th2 immune response is a systemic immune response.

104. The pharmaceutical composition of claim 100, wherein the antigen is not an self antigen.

105. The pharmaceutical composition of claim 100, wherein the Th2-immunostimulatory nucleic acid is formulated in a delivery vehicle selected from the group consisting of bioadhesive polymers, cochleates, dendrimers, enteric-coated capsules, emulsomes, ISCOMs, liposomes, cationic lipids, microspheres, nanospheres, polymer rings, proteosomes, and virosomes.

106. The pharmaceutical composition of claim 100, further comprising a therapeutic agent.

107. The pharmaceutical composition of claim 106, wherein the therapeutic agent is a Th1 adjuvant.

108. The pharmaceutical composition of claim 106, wherein the therapeutic agent is a Th2 adjuvant.

109. The pharmaceutical composition of claim 106, wherein the therapeutic agent is a cytokine.

110. The pharmaceutical composition of claim 106, wherein the therapeutic agent is a drug for treating Th1 mediated disorders.

111. The pharmaceutical composition of claim 105, wherein the Th2-immunostimulatory nucleic acid and antigen are present in different delivery vehicles.

112. A pharmaceutical composition, comprising:
an effective amount of a Th2 immunostimulatory nucleic acid for stimulating a Th2 immune response when administered mucosally or dermally, and an adjuvant, in a pharmaceutically acceptable carrier.

113. The pharmaceutical composition of claim 112, wherein the Th2 immune response is a mucosal immune response.

114. The pharmaceutical composition of claim 112, wherein the Th2 immune response is a systemic immune response.

115. The pharmaceutical composition of claim 112, wherein the adjuvant is a Th1 adjuvant.

116. The pharmaceutical composition of claim 112, wherein the Th1 adjuvant is selected from the group consisting of CpG nucleic acids, MF59, SAF, MPL, and QS21.

117. The pharmaceutical composition of claim 112, wherein the adjuvant is a Th2 adjuvant.

118. The pharmaceutical composition of claim 117, wherein the Th2 adjuvant is selected from the group consisting of adjuvants that creates a depot effect, adjuvants that stimulate the immune system, adjuvants that create a depot effect and stimulate the immune system and mucosal adjuvants.

119. The pharmaceutical composition of claim 118, wherein the adjuvant that creates a depot effect is selected from the group consisting of alum; emulsion-based formulations including mineral oil, non-mineral oil, water-in-oil or oil-in-water-in oil emulsion, oil-in-water emulsions such as Seppic ISA series of Montanide adjuvants; and PROVAX.

120. The pharmaceutical composition of claim 118, wherein the adjuvant that stimulates the immune system is selected from the group consisting of saponins purified from the bark of the Q. saponaria tree; poly[di(carboxylatophenoxy)phosphazene; derivatives of lipopolysaccharides, muramyl dipeptide and threonyl-muramyl dipeptide; OM-174; and Leishmania elongation factor.

121. The pharmaceutical composition of claim 118, wherein the adjuvant that creates a depot effect and stimulates the immune system is selected from the group consisting of ISCOMs; SB-AS2; SB-AS4; non-ionic block copolymers that form micelles such as CRL 1005; and Syntex Adjuvant Formulation.

122. The pharmaceutical composition of claim 118, wherein the mucosal adjuvant is selected from the group consisting of CpG nucleic acids, Bacterial toxins, Cholera toxin, CT derivatives, CT B subunit; CTD53; CTK97; CTK104; CTD53/K63; CTH54; CTN107; CTE114; CTE112K; CTS61F; CTS106; and CTK63, Zonula occludens toxin, zot, Escherichia coli heat-labile enterotoxin, Labile Toxin, LT derivatives, LT B subunit; LT7K; LT61F; LT112K; LT118E; LT146E; LT192G; LTK63; and LTR72, Pertussis toxin, PT-9K/129G; Toxin derivatives; Lipid A derivatives, MDP derivatives; Bacterial outer membrane proteins, outer surface protein A (OspA) lipoprotein of Borrelia burgdorferi, outer membrane protein of Neisseria meningitidis; Oil-in-water emulsions, Aluminum salts; and Saponins, ISCOMs, the Seppic ISA series of Montanide adjuvants, Montanide ISA 720; PROVAX; Syntext Adjuvant Formulation; poly[di(carboxylatophenoxy) phosphazene and Leishmania elongation factor.

123. The pharmaceutical composition of claim 112, further comprising a therapeutic agent selected from the group consisting of an anti-viral agent, an anti-bacterial agent, an anti-parasitic agent, an anti-fungal agent, and a cancer medicament.

124. A method for treating an infectious disease in a subject, comprising:
administering to a subject having an infectious disease a Th2 immunostimulatory nucleic acid in an amount effective to treat the infectious disease when administered mucosally, dermally, or parenterally, wherein the subject has not been exposed to a Th1 immunostimulatory nucleic acid.

125. The method of claim 124, wherein the infectious disease is not an extracellular infection.

126. The method of claim 124, wherein the method is a method for treating a viral infection.

127. The method of claim 126, further comprising, administering an anti-viral agent.

128. The method of claim 124, wherein the method is a method for treating or preventing a bacterial infection.

129. The method of claim 128, further comprising, administering an anti-bacterial agent.

130. The method of claim 124, wherein the method is a method for treating or preventing a parasitic infection.

131. The method of claim 130, further comprising administering an anti-parasitic agent.

132. The method of claim 124, wherein the Th2 immunostimulatory nucleic acid is administered mucosally.

133. The method of claim 124, wherein the Th2 immunostimulatory nucleic acid is administered locally.

134. The method of claim 124, wherein the Th2 immunostimulatory nucleic acid is administered parenterally.

135. A method of preventing an infectious disease in a subject, comprising administering to a subject at risk of developing an infectious disease a Th2 immunostimulatory nucleic acid in an amount effective to prevent the infectious disease when administered mucosally, dermally, or parenterally, wherein the subject has not been exposed to a Th1 immunostimulatory nucleic acid.

136. A method for treating or preventing a cancer in a subject, comprising:
administering to a subject having a cancer or at risk of developing a cancer a Th2 immunostimulatory nucleic acid in an amount effective to treat or prevent the cancer when administered mucosally, dermally, or parenterally.

137. The method of claim 136, wherein the cancer is a cancer selected from the group consisting of oral cavity cancer, throat cancer, stomach cancer, colon cancer, rectal cancer, cervical cancer.

138. The method of claim 136, wherein the Th2-immunostimulatory nucleic acid is administered mucosally.

139. The method of claim 136, wherein the Th2-immunostimulatory nucleic acid is administered locally.

140. The method of claim 136, wherein the Th2-immunostimulatory nucleic acid is administered parenterally.

141. The method of claim 136, further comprising administering an anti-cancer agent.

142. A method for stimulating an antibody dependent cellular cytotoxic (ADCC) immune response in a subject, comprising administering to the subject a Th2 immunostimulatory nucleic acid and an antibody in an effective amount for inducing ADCC.

143. The method of claim 142, wherein the antibody is a monoclonal antibody.

144. The method of claim 142, wherein the monoclonal antibody is selected from the group consisting of Rituxan, IDEC-C2B8, anti-CD20 Mab, Panorex, 3622W94, anti-EGP40 (17-1A) pancarcinoma antigen on adenocarcinomas Herceptin, anti-Her2, Anti-EGFr, BEC2, anti-idiotypic-GD₃ epitope, Ovarex, B43.13, anti-idiotypic CA125, 4B5, Anti-VEGF, RhuMAb, MDX-210, anti-HER-2, MDX-22, MDX-220, MDX-447, MDX-260, anti-GD-2, Quadramet, CYT-424, IDEC-Y2B8, Oncolym, Lym-1, SMART M195, ATRAGEN, LDP-03, anti-CAMPATH, ior t6, anti CD6, MDX-11, OV103, Zenapax, Anti-Tac, anti-IL-2 receptor, MELIMMUNE-2, MELIMMUNE-1, CEACIDE, Pretarget, NovoMAb-G2, TNT, anti-histone, Gliomab-H, GNI-250, EMD-72000, LymphoCide, CMA 676, Monopharm-C, ior egf/r3, ior c5, anti-FLK-2, SMART 1D10, SMART ABL 364, and ImmuRAIT-CEA.

145. The method of claim 142, wherein the subject has a disorder selected from the group consisting of cancer, and infectious disease.

146. The method of claim 142, wherein the Th2 immunostimulatory nucleic acid is not conjugated to the antibody.

147. The method of claim 142, wherein the subject has a cancer.

148. The method of claim 147, further comprising administering radiation or chemotherapy to the subject.

149. The method of claim 148, wherein the chemotherapy is selected from the group consisting of Taxol, cisplatin, doxorubicin, and adriamycin.

150. A pharmaceutical composition, comprising:
a Th2 immunostimulatory nucleic acid in an effective amount for inducing ADCC, a monoclonal antibody, and a pharmaceutically acceptable carrier.

151. The composition of claim 150, wherein the monoclonal antibody is selected from the group consisting of Rituxan, IDEC-C2B8, anti-CD20 Mab, Panorex, 3622W94, anti-EGP40 (17-1A) pancarcinoma antigen on adenocarcinomas Herceptin, anti-Her2, Anti-EGFr, BEC2, anti-idiotypic-GD₃ epitope, Ovarex, B43.13, anti-idiotypic CA125, 4B5, Anti-VEGF, RhuMAb, MDX-210, anti-HER-2, MDX-22, MDX-220, MDX-447, MDX-260, anti-GD-2, Quadramet, CYT-424, IDEC-Y2B8, Oncolym, Lym-1, SMART M195, ATRAGEN, LDP-03, anti-CAMPATH, ior t6, anti CD6, MDX-11, OV103, Zenapax, Anti-Tac, anti-IL-2 receptor, MELIMMUNE-2, MELIMMUNE-1, CEACIDE, Pretarget, NovoMAb-G2, TNT, anti-histone, Gliomab-H, GNI-250, EMD-72000, LymphoCide, CMA 676, Monopharm-C, ior egf/r3, ior c5, anti-FLK-2, SMART 1D10, SMART ABL 364, and ImmuRAIT-CEA.

152. A composition, comprising:
a Th2 immunostimulatory nucleic acid having a phosphodiester backbone, formulated in a delivery vehicle selected from the group consisting of bioadhesive polymers, enteric-coated capsules, microspheres, nanospheres, and polymer rings.

153. The composition of claim 152, wherein the Th2 immunostimulatory nucleic acid is formulated for mucosal delivery.
